(19) United States
(12) Patent Application Publication
(10) Pub. No.: US 2020/0245441 A1
(43) Pub. Date: Jul. 30, 2020

(54) RADIATION IMAGING APPARATUS, OPERATION METHOD THEREFOR, AND COMPUTER-READABLE MEDIUM

(71) Applicant: CANON KABUSHIKI KAISHA, Tokyo (JP)

(72) Inventors: Akira Tsukuda, Kawasaki-shi (JP); Atsushi Iwashita, Tokyo (JP); Kosuke Terui, Yokohama-shi (JP); Sota Torii, Kawasaki-shi (JP)

(21) Appl. No.: 16/847,074

(22) Filed: Apr. 13, 2020

Related U.S. Application Data
(63) Continuation of application No. PCT/JP2018/037867, filed on Oct. 11, 2018.

(30) Foreign Application Priority Data
Oct. 16, 2017 (JP) 2017-200574

(51) Int. Cl. H05G 1/26; G01N 23/04

(52) U.S. Cl. CPC H05G 1/26; A61B 6/5205; G01N 2223/304; G01N 23/04

(57) ABSTRACT

Provided is a radiation imaging apparatus configured to image an imaging target object through use of a radiation generated by a radiation generator arranged to generate the radiation. The radiation imaging apparatus includes a spectrum calculating unit configured to calculate a spectrum of the radiation based on a transient response characteristic of the radiation generator.

### CROSS-REFERENCE TO RELATED APPLICATIONS

This application is a Continuation of International Patent Application No. PCT/JP2018/037867, filed Oct. 11, 2018, which claims the benefit of Japanese Patent Application No. 2017-200574, filed Oct. 16, 2017, both of which are hereby incorporated by reference herein in their entirety.

### BACKGROUND OF THE INVENTION

### Field of the Invention

The present invention relates to a radiation imaging apparatus configured to image an imaging target object through use of radiation, an operation method therefor, and a computer-readable medium having stored thereon a program for causing a computer to execute the operation method.

### Description of the Related Art

In recent years, as one of the technical fields of a radiation imaging apparatus, a spectral imaging technology being an imaging technology using radiation energy information has been widely studied and put to practical use. In the spectral imaging, it is required to accurately calculate a spectrum of radiation. For example, in “An accurate method for computer-generating tungsten anode x-ray spectra from 30 to 140 kV”, John M. Boone and J. Anthony Seibert, Medical Physics 24 (11), page 1,661, 1997, there is described a technology for calculating a spectrum from a tube voltage being one of setting parameters of a radiation generator. In Japanese Patent Application Laid-Open No. 2013-208486, there is also described a technology for sampling a tube voltage waveform of a radiation source by an analog/digital (A/D) converter (specifically, sampling the tube voltage waveform by convolving an analog signal with a weighting function g* representing the sampling characteristic of the A/D converter) to calculate a spectrum from the tube voltage waveform.

However, there have been some cases in which, when the method described in “An accurate method for computer-generating tungsten anode x-ray spectra from 30 to 140 kV”, John M. Boone and J. Anthony Seibert, Medical Physics 24 (11), page 1,661, 1997 is employed, the shape of the calculated spectrum deviates from the shape of a spectrum actually measured by a spectrometer. In particular, when radiation imaging is performed on the heart or another such rapidly moving imaging target object, the pulse width of the radiation tends to be kept at as small a value as possible in order to reduce a sense of discomfort of the image, and hence, a deviation between the shape of the actually measured spectrum and the shape based on the calculated value tends to increase at that time.

### SUMMARY OF THE INVENTION

The present invention has been made in view of such a problem, and provides a mechanism capable of calculating a spectrum of a radiation with high accuracy.

According to at least one embodiment of the present invention, there is provided a radiation imaging apparatus configured to image an imaging target object through use of radiation generated by a radiation generator arranged to generate the radiation, the radiation imaging apparatus including: a spectrum calculating unit configured to calculate a spectrum of the radiation based on a transient response characteristic of the radiation generator.

According to at least one embodiment of the present invention, there are also provided an operation method for the above-mentioned radiation imaging apparatus and a computer-readable medium having stored thereon a program for causing a computer to execute the operation method.

Further features of the present invention will become apparent from the following description of exemplary embodiments with reference to the attached drawings.

### BRIEF DESCRIPTION OF THE DRAWINGS

FIG. 1A is a diagram for illustrating an example of a schematic configuration of a radiation imaging apparatus according to a first embodiment of the present invention.

FIG. 1B is a diagram for illustrating an example of a hardware configuration of a processing/control unit illustrated in FIG. 1A.

FIG. 2 is a diagram for illustrating an example of a circuit configuration of a radiation detector illustrated in FIG. 1A.

FIG. 3A is a graph for showing an example of an operation method for the radiation detector illustrated in FIG. 1A.

FIG. 3B shows an example of a tube voltage waveform relating to a tube voltage change of a radiation generator.

FIG. 4 is a flow chart for illustrating an example of a processing procedure for an operation method for the radiation imaging apparatus according to the first embodiment of the present invention.

FIG. 5 is a diagram for illustrating an example of a functional configuration of a spectrum calculating unit relating to a first acquisition example of acquiring the tube voltage waveform of the radiation generator illustrated in FIG. 1A in the first embodiment of the present invention.

FIG. 6 is a diagram for illustrating an example of a functional configuration of a waveform processing unit illustrated in FIG. 5.

FIG. 7 is a flow chart for illustrating an example of a processing procedure of a waveform processing unit illustrated in FIG. 6.

FIG. 8 is a diagram for illustrating an example of a functional configuration of the spectrum calculating unit relating to a second acquisition example of acquiring the tube voltage waveform of the radiation generator illustrated in FIG. 1A in the first embodiment of the present invention.

FIG. 9 is a flow chart for illustrating an example of a processing procedure of an imaging condition acquiring unit and a tube voltage deriving unit illustrated in FIG. 8.

FIG. 10A is an explanatory graph for showing processing of an interpolation/extrapolation unit shown in FIG. 8.

FIG. 10B is an explanatory graph for showing processing of the interpolation/extrapolation unit shown in FIG. 8.

FIG. 10C is an explanatory graph for showing processing of the interpolation/extrapolation unit shown in FIG. 8.

FIG. 11 is a diagram for illustrating an example of a functional configuration of the spectrum calculating unit relating to a third acquisition example of acquiring the tube voltage waveform of the radiation generator illustrated in FIG. 1A in the first embodiment of the present invention.

FIG. 12 is a flow chart for illustrating an example of a processing procedure of a tube voltage deriving unit illustrated in FIG. 11.

FIG. 13 is graphs for showing an example of calculating an impulse response by an impulse response calculating unit illustrated in FIG. 11.

FIG. 14 is a flow chart for illustrating an example of a processing procedure for an impulse response calibration method relating to the third acquisition example of acquiring the tube voltage waveform of the radiation generator illustrated in FIG. 1A in the first embodiment of the present invention.

FIG. 15 is a diagram for illustrating an example of a functional configuration of the spectrum calculating unit relating to a fourth acquisition example of acquiring the tube voltage waveform of the radiation generator illustrated in FIG. 1A in the first embodiment of the present invention.

FIG. 16 is an explanatory graph for showing processing relating to the fourth acquisition example of acquiring the tube voltage waveform of the radiation generator illustrated in FIG. 1A in the first embodiment of the present invention.

FIG. 17A is a flow chart for illustrating an example of a processing procedure for an impulse response calibration method relating to the fourth acquisition example of acquiring the tube voltage waveform of the radiation generator illustrated in FIG. 1A in the first embodiment of the present invention.

FIG. 17B is a diagram for showing a mathematical expression relating to an impulse response and a mathematical expression relating to a step response.

FIG. 18 is a diagram for illustrating an example of a functional configuration of the spectrum calculating unit illustrated in FIG. 1A in a second embodiment of the present invention.

FIG. 19 is a flow chart for illustrating an example of a processing procedure of a spectrum calculating unit illustrated in FIG. 18.

FIG. 20 is explanatory graphs for showing processing of the spectrum calculating unit illustrated in FIG. 18.

### DESCRIPTION OF THE EMBODIMENTS

Exemplary embodiments of the present invention will now be described in detail in accordance with the accompanying drawings.

### First Embodiment

First, a first embodiment of the present invention is described.

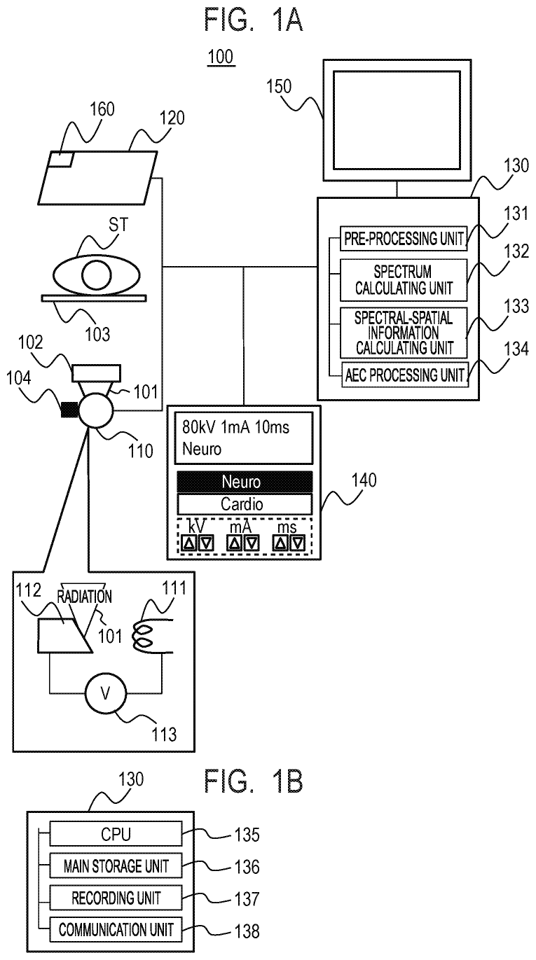

FIG. 1A and FIG. 1B are diagrams for illustrating an example of a schematic configuration of a radiation imaging apparatus 100 according to the first embodiment of the present invention.

The radiation imaging apparatus 100 is an apparatus configured to image an imaging target object ST through use of radiation 101. As illustrated in FIG. 1A, the radiation imaging apparatus 100 includes a radiation generator 110, a radiation detector 120, a processing/control unit 130, an operating unit 140, a display unit 150, a measuring device 160, a radiation filter 102, a stand 103, and a thermometer 104.

The radiation generator 110 is an apparatus arranged to generate the radiation 101 toward the imaging target object ST, and is a radiation tube bulb in the first embodiment. The description of the first embodiment is directed to a case in which the radiation generator 110 is a radiation tube bulb, but the present invention is not limited to this form, and the form of a configuration including not only the radiation tube bulb but also another component as the radiation generator 110 is also applicable to the present invention.

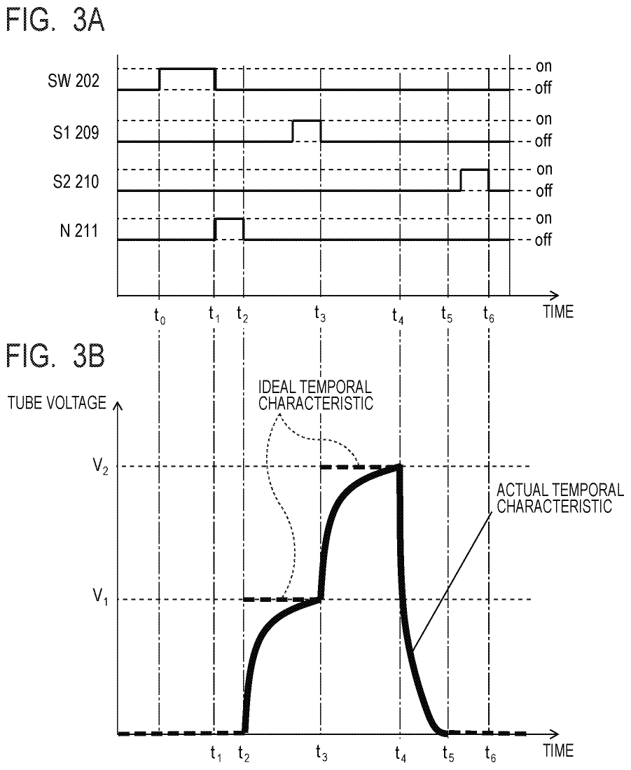

As illustrated in FIG. 1A, the radiation tube bulb being the radiation generator 110 includes a cathode 111 arranged to generate thermoelectrons, an anode 112 serving as a target, and a voltmeter 113 for measuring voltages of the cathode 111 and the anode 112. In the first embodiment, a tube voltage waveform of the radiation tube bulb being the radiation generator 110 can be obtained by constantly monitoring a difference in potential between the anode 112 and the cathode 111 through use of, for example, the voltmeter 113. There are two kinds of tube voltages to be set. That is, a tube voltage corresponding to the low-energy radiation 101 (first tube voltage V₁ shown in FIG. 3B) and a tube voltage corresponding to the high-energy radiation 101 (second tube voltage V₂ shown in FIG. 3B)) are set.

In the example illustrated in FIG. 1A, the radiation 101 emitted from the radiation generator 110 enters the imaging target object ST through the radiation filter 102 and the stand 103 on which the imaging target object ST is placed. In the first embodiment, the stand 103 is formed of a member that transmits the radiation 101 therethrough.

The radiation detector 120 is an apparatus arranged at a position opposed to the radiation generator 110 across the imaging target object ST and arranged to detect the radiation 101 generated by the radiation generator 110 (desirably, the radiation 101 transmitted through the imaging target object ST). Specifically, the radiation detector 120 detects the incoming radiation 101 as an image signal being an electric signal.

The processing/control unit 130 controls each component of the radiation imaging apparatus 100 to centrally control the operation of the radiation imaging apparatus 100, and performs various kinds of processing. In the first embodiment, the processing/control unit 130 is, for example, a computer. As illustrated in FIG. 1A, the processing/control unit 130 includes functional components of a pre-processing unit 131, a spectrum calculating unit 132, a spectral-spatial information calculating unit 133, and an automatic exposure control (AEC) processing unit 134.

The pre-processing unit 131 performs offset correction processing, gain correction, defect correction, and other such pre-processing on the image signal generated by the detection processing of the radiation detector 120.

The spectrum calculating unit 132 calculates a spectrum of the radiation 101 based on the transient response characteristic of the radiation generator 110. This spectrum indicates, for example, a relationship between energy and radiation intensity. The spectrum may indicate a relationship between energy and the number of photons or radiation intensity.

The spectral-spatial information calculating unit 133 calculates spectral-spatial information being spatial information on the spectrum of the radiation 101 based on: an image based on the image signal generated by the detection processing of the radiation detector 120 (more desirably, the image signal pre-processed by the pre-processing unit 131); and the spectrum of the radiation 101 calculated by the spectrum calculating unit 132.

The AEC processing unit 134 performs processing for determining, for example, the pulse width of the radiation 101 actually emitted from the radiation generator 110. At this time, the pulse width of the radiation 101 is determined by comparing the pulse width of the radiation 101 set by an operation of the operating unit 140 with the pulse width of the radiation 101 calculated from, for example, the pixel values of the above-mentioned image. The AEC processing unit 134 also performs processing for determining, for example, the timing of sampling and holding based on the determined pulse width of the radiation 101.

The operating unit 140 is to be operated by an operator of the radiation imaging apparatus 100 when performing operation input to the radiation imaging apparatus 100. The use of the operating unit 140 allows the operator to designate, for example, imaging conditions relating to radiation imaging. For example, through the operating unit 140, the operator can designate the tube voltage (for example, designated in kilovolts (kV)) of the radiation tube bulb being the radiation generator 110, the tube current (for example, designated in milliamperes (mA)) of the radiation tube bulb, and the pulse width (for example, designated in milliseconds (ms)) of the radiation 101. The operator can also designate, for example, the kind of the radiation filter 102, the arrangement of the stand 103, and a body part to be imaged (e.g., Neuro or Cardio) relating to the imaging target object ST through use of the operating unit 140. In addition, when the body part to be imaged is designated, the tube voltage and the tube current of the radiation tube bulb, the pulse width of the radiation 101, and the arrangement of the radiation filter 102 and the stand 103 are set in correspondence with the designated body part to be imaged.

The display unit 150 displays, for example, various images and various kinds of information based on the control of the processing/control unit 130. Examples to be displayed on the display unit 150 include the spectral-spatial information calculated by the spectral-spatial information calculating unit 133.

The measuring device 160 is provided near a part of the radiation detector 120 (in the example illustrated in FIG. 1A, an upper part of the radiation detector 120), and includes a radiation dosimeter for measuring the dose of the radiation 101 and a spectrometer for measuring a spectrum of the radiation 101. The measuring device 160 is used for acquiring a tube voltage waveform and a spectrum of the radiation 101, which are described later.

The thermometer 104 is an apparatus provided near the radiation generator 110, for measuring a temperature of the radiation generator 110. The transient response characteristic of the radiation tube bulb being the radiation generator 110 changes depending on the temperature as well, and hence it is possible to calculate a more optimal transient response characteristic by providing the thermometer 104. In the radiation imaging apparatus 100 according to the first embodiment, instead of installing the thermometer 104, it is possible to calculate, for example, an amount (e.g., a heat unit) relating to energy applied to the radiation tube bulb, to thereby estimate the temperature characteristic of the radiation tube bulb.

FIG. 1B is a diagram for illustrating an example of a hardware configuration of the processing/control unit 130 (for example, a computer) illustrated in FIG. 1A. As illustrated in FIG. 1B, the processing/control unit 130 includes hardware components of a central processing unit (CPU) 135, a main storage unit 136, a recording unit 137, and a communication unit 138. In this case, the communication unit 138 is a communication unit for a local area network (LAN), serial rapid input/output (sRIO (trademark)), or another such network architecture.

Now, an example of a correspondence relationship between the functional components of the processing/control unit 130 illustrated in FIG. 1A and the hardware components of the processing/control unit 130 illustrated in FIG. 1B is described. For example, the pre-processing unit 131, the spectrum calculating unit 132, the spectral-spatial information calculating unit 133, and the AEC processing unit 134, which are illustrated in FIG. 1A, are implemented by the CPU 135, programs stored in the main storage unit 136, and the communication unit 138, which are illustrated in FIG. 1B.

The radiation imaging apparatus 100 according to the first embodiment may be provided with, for example, a high voltage generating apparatus for the radiation 101, a radiation diaphragm, and a C-arm or another such supporting mechanism for the radiation generator 110 and the radiation detector 120.

Now, the spectral-spatial information calculated by the spectral-spatial information calculating unit 133 is described. As an example of the spectral-spatial information calculated by the spectral-spatial information calculating unit 133, there is a spatial distribution of the thickness of each substance. Assuming that an image obtained when the imaging target object ST is not present on the stand 103 is represented by I₀, and an image obtained when the imaging target object ST is present on the stand 103 is represented by I, I/I₀ is expressed by Expression (1).

$$I / {I}_{0} = \frac{{{\int }^{∞}}_{0} N ( E ) exp ( - {μ}_{1} ( E ) {d}_{1} - {μ}_{2} ( E ) {d}_{2} \} EdE}{{{\int }^{∞}}_{0} N ( E ) EdE} \qquad ( 1 )$$

In Expression (1), E represents energy, N(E) represents the spectrum of the radiation 101, μ₁(E) represents the linear attenuation coefficient of a substance 1, μ₂(E) represents the linear attenuation coefficient of a substance 2, d₁ represents the thickness of the substance 1, and d₂ represents the thickness of the substance 2. The unknown variables in Expression (1) are the thickness d₁ and the thickness d₂. When radiations 101 having two different kinds of energy are used for imaging to substitute their values into Expression (1), two independent expressions can be derived. Therefore, it is possible to obtain the values of the thickness d₁ and the thickness d₂ by solving the two independent expressions.

As another example of the spectral-spatial information, there is a spatial distribution of an effective atomic number and an areal density. The spatial distribution of the effective atomic number and the areal density can be expressed by Expression (2).

$$I / {I}_{0} = \frac{{{\int }^{∞}}_{0} N ( E ) exp ( - μ ( {Z}_{eff} , E ) {D}_{eff} \} EdE}{{{\int }^{∞}}_{0} N ( E ) EdE} \qquad ( 2 )$$

In Expression (2), E represents energy, N(E) represents the spectrum of the radiation 101, μ(Z_{eff}, E) represents the mass attenuation coefficient at an effective atomic number Z_{eff} and the energy E, and D_{eff} represents an effective areal density. The unknown variables in Expression (2) are the effective atomic number Z_{eff} and the effective areal density D_{eff}. Therefore, as in the case of obtaining the spatial distribution of the thickness of each substance, when radiations 101 having two different kinds of energy are used for imaging to substitute their values into Expression (2), two independent expressions can be derived. Therefore, it is possible to obtain the values of the effective atomic number Z_{eff} and the effective areal density D_{eff} by solving the two independent expressions.

In both Expression (1) and Expression (2), the spectral-spatial information is calculated through use of the spectrum N(E) of the radiation 101. Therefore, in order to calculate the spectral-spatial information with high accuracy, it is required to calculate the spectrum N(E) of the radiation 101 with high accuracy. In the first embodiment, the form of emitting the radiations 101 having two different kinds of energy is described, but the present invention is not limited to this form, and a form of emitting the radiations 101 having three or more different kinds of energy is also applicable to the present invention. When this form is employed, it is possible to increase, for example, the number of substances to be separated. However, when this form is employed, it is required to increase the number of sample-and-hold circuits provided to the radiation detector 120 to four or more as the requirement arises.

Next, a circuit configuration of the radiation detector 120 illustrated in FIG. 1A and an operation method therefor are described.

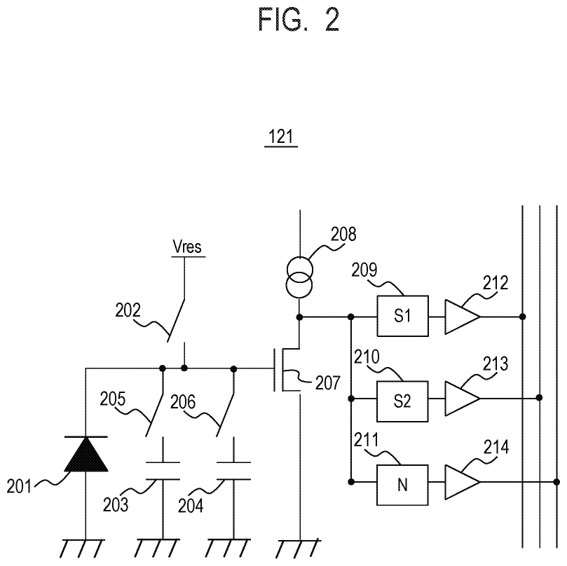

FIG. 2 is a diagram for illustrating an example of the circuit configuration of the radiation detector 120 illustrated in FIG. 1A. Specifically, FIG. 2 is an illustration of an example of a circuit configuration of one pixel 121 among a plurality of pixels for detecting the radiation 101, which are provided to the radiation detector 120.

The pixel 121 illustrated in FIG. 2 generates an image signal corresponding to the application of the radiation 101. The pixel 121 includes a photodiode 201, switches 202, 205, and 206, capacitors 203 and 204, a source follower circuit 207, a constant current source 208, sample-and-hold circuits 209, 210, and 211, and output amplifiers 212, 213, and 214. The pixel 121 illustrated in FIG. 2 is also provided with a scintillator (not shown) for converting the radiation 101 into light, for example, on a side from which the radiation 101 enters.

The photodiode 201 is a photoelectric conversion unit arranged to convert light from a scintillator (not shown) into a charge being an electric signal. The switch 202 is a switch for resetting the charge in the pixel 121. The capacitors 203 and 204 are capacitors for changing the sensitivity of the pixel 121. The switches 205 and 206 are switches for selecting whether or not to change the sensitivity of the pixel 121. The source follower circuit 207 is a circuit for reading the voltage value of the pixel 121.

The sample-and-hold circuit 209 (first signal holding unit) is a first signal holding unit configured to hold a first image signal output based on charges accumulated in the photodiode 201 under a state in which the energy of the applied radiation 101 is low. The sample-and-hold circuit 210 (second signal holding unit) is a second signal holding unit configured to hold a second image signal output based on charges accumulated in the photodiode 201 under a state in which the energy of the applied radiation 101 is high. The sample-and-hold circuit 211 is a third signal holding unit configured to hold a noise signal. Through provision of the sample-and-hold circuit 211, it is possible to subtract noise included in the sample-and-hold circuit 209 holding the first image signal and noise included in the sample-and-hold circuit 210 holding the second image signal.

Further, through provision of two capacitors 203 and 204, it is possible to select more combinations of gains. In this case, a state of the pixel 121 in which the capacitors 203 and 204 are not connected is referred to as “high gain state”, a state of the pixel 121 in which only the capacitor 203 is connected is referred to as “mid-gain state”, and a state of the pixel 121 in which both the capacitors 203 and 204 are connected is referred to as “low gain state”.

FIG. 3A and FIG. 3B are graphs for showing an example of an operation method for the radiation detector 120 illustrated in FIG. 1A. Specifically, FIG. 3A shows a timing chart for showing an example of an operation method for the pixel 121 illustrated in FIG. 2, and FIG. 3B shows an example of a tube voltage waveform relating to a tube voltage change of the radiation generator 110 corresponding to the timing chart shown in FIG. 3A.

In FIG. 3A, SW_202 indicates a state of the switch 202 illustrated in FIG. 2. S1_209 indicates a holding timing of the sample-and-hold circuit 209 illustrated in FIG. 2, and S2_210 indicates a holding timing of the sample-and-hold circuit 210 illustrated in FIG. 2. N_211 indicates a holding timing of the sample-and-hold circuit 211 illustrated in FIG. 2.

Now, the timing chart shown in FIG. 3A is described. First, at a time to, for example, the processing/control unit 130 turns on the switch 202 indicated by SW_202 to set the voltage of the pixel 121 to a reset voltage Vres. Subsequently, at a time t₁, for example, the processing/control unit 130 turns on the sample-and-hold circuit 211(N) indicated by N_211 to sample and hold the value of the noise. After that, at a time t₂, for example, the processing/control unit 130 turns off the sample-and-hold circuit 211(N) indicated by N_211 to end the sampling and holding of the value of the noise. With this control, charge accumulation based on the detection of the radiation 101 is started from the time t₂.

Subsequently, between the time t₂ and a time t₃, for example, the processing/control unit 130 controls the radiation generator 110 to generate the low-energy radiation 101 corresponding to the first tube voltage V₁ shown in FIG. 3B. In this case, at the time t₃, for example, the processing/control unit 130 turns on the sample-and-hold circuit 209 indicated by S1_209 to sample and hold a value corresponding to the first tube voltage V₁.

Subsequently, between the time t₃ and a time t₅, for example, the processing/control unit 130 controls the radiation generator 110 to generate the high-energy radiation 101 corresponding to the second tube voltage V₂ shown in FIG. 3B. In this case, the tube voltage waveform shown in FIG. 3B reaches a local maximum value at a time t₄ (the set pulse width is often set to a difference between the time t₄ and the time t₂). After that, at the time t₅, for example, the processing/control unit 130 ends the application of the radiation 101.

Subsequently, at a time t₆, for example, the processing/control unit 130 turns on the sample-and-hold circuit 210 indicated by S2_210 to sample and hold a value including data on the second tube voltage V₂.

After that, the processing/control unit 130 subtracts the value of the noise N accumulated in the sample-and-hold circuit 209 indicated by S1_209 to obtain a value of S1-N, and also subtracts the value of the noise N accumulated in the sample-and-hold circuit 210 indicated by S2_210 to obtain a value of S2-N. Charges derived from both the low-energy radiation 101 and the high-energy radiation 101 are accumulated in the value of S2-N, but the value of S1-N is further subtracted from the value of S2-N, to thereby be able to extract the charges accumulated only between the time t₃ and the time t₅.

Against this backdrop, the inventor of the present invention has found that, with respect to the tube voltage waveform input to the radiation tube bulb being the radiation generator 110, the tube voltage actually observed is delayed based on the transient response characteristic of the radiation tube bulb, and that a spectrum of the radiation also changes depending on the transient response characteristic of the radiation tube bulb. A method of calculating the spectrum in consideration of the transient response characteristic is described below.

In the tube voltage waveform shown in FIG. 3B, the temporal characteristics of the first tube voltage V₁ and the second tube voltage V₂ each require a finite time period until the set tube voltage is reached due to the transient response characteristic of the radiation tube bulb being the radiation generator 110 (“actual temporal characteristics” shown in FIG. 3B). This causes the shape of the actually measured spectrum of the radiation 101 to greatly differ from the shape based on values expected from the first tube voltage V₁ and the second tube voltage V₂. Specifically, for example, the shape of the spectrum expected at a given tube voltage is calculated assuming that the tube voltage is constant with respect to time, and the spectrum is calculated with accuracy when the tube voltage exhibits a response characteristic as shown in “ideal temporal characteristic” of FIG. 3B.

In view of the above-mentioned point, in the first embodiment, the spectrum calculating unit 132 calculates the spectrum of the radiation 101 based on the transient response characteristic of the radiation generator 110 calculated by actual measurement or simulation. Specifically, the first embodiment employs a form in which the spectrum calculating unit 132 derives the tube voltage waveform of the radiation generator 110 based on the transient response characteristic of the radiation generator 110, and calculates the spectrum of the radiation 101 through conversion based on the derived tube voltage waveform.

Next, a processing procedure for an operation method for the radiation imaging apparatus 100 according to the first embodiment is described.

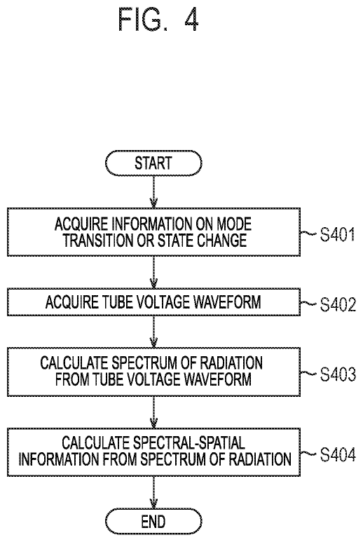

FIG. 4 is a flow chart for illustrating an example of the processing procedure for the operation method for the radiation imaging apparatus 100 according to the first embodiment of the present invention.

First, in Step S401, the processing/control unit 130 causes a transition in mode of the radiation tube bulb being the radiation generator 110. In this case, the “mode” of the radiation detector 120 includes: modes to be effected by the operator, which includes the tube voltage (for example, designated in kilovolts (kV)), the tube current (for example, designated in milliamperes (mA)), the pulse width of the radiation 101 (for example, designated in milliseconds (ms)), changes in positions of the radiation filter 102, the stand 103, and a fixing jig, and a change in body part to be imaged (as described above, when the body part to be imaged is designated, the tube voltage and the tube current of the radiation tube bulb, the pulse width of the radiation 101, and the arrangement of the radiation filter 102 and the stand 103 are set in correspondence with the designated body part to be imaged); and a mode to be effected by the AEC processing unit 134, which includes a change in pulse width of the radiation 101. Transition from one mode to another mode among those modes is referred to as “mode transition”. In addition, when a state change of the radiation imaging apparatus 100 is detected, the spectrum of the radiation 101 is recalculated in accordance with the flow chart illustrated in FIG. 4. In this case, the “state change” of the radiation imaging apparatus 100 includes the temperature and the heat unit of the radiation tube bulb being the radiation generator 110, and corresponds to, for example, a case in which the temperature of the radiation tube bulb being monitored by the thermometer 104 exceeds a given threshold value or a case in which the heat unit of the radiation tube bulb exceeds a given threshold value. When detecting a mode transition or a state change, the processing/control unit 130 acquires information on the detected mode transition or the detected state change, and uses the information for later calculation processing of Step S403 or Step S404. At this time, the processing/control unit 130 simultaneously acquires information on a spectrum that does not vary with time, for example, information on the radiation generator 110 (information on, for example, the anode 112 serving as a target and the inherent filtration of the radiation tube bulb).

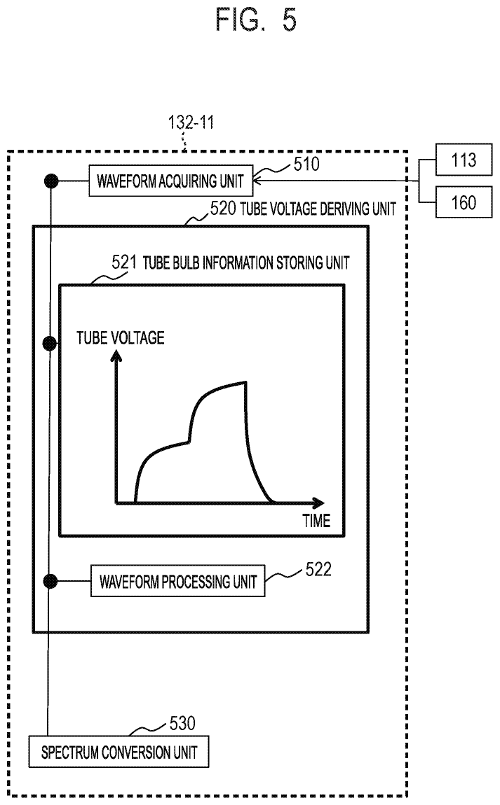

Subsequently, in Step S402, the processing/control unit 130 (for example, the spectrum calculating unit 132) acquires the tube voltage waveform of the radiation tube bulb being the radiation generator 110. Examples of a method of acquiring the tube voltage waveform of the radiation generator 110 include a method of acquiring and processing a value output from the voltmeter 113 and a method of performing the calculation based on information stored in a tube bulb information storing unit of the spectrum calculating unit 132. A specific method of acquiring the tube voltage waveform of the radiation generator 110 in the first embodiment is described later with reference to FIG. 5 to FIG. 17B.

Subsequently, in Step S403, the spectrum calculating unit 132 calculates the spectrum of the radiation 101 through conversion based on the tube voltage waveform of the radiation generator 110 acquired in Step S402.

Now, a specific processing example of Step S403 is described. According to “An accurate method for computer-generating tungsten anode x-ray spectra from 30 to 140 kV”, John M. Boone and J. Anthony Seibert, Medical Physics 24 (11), page 1,661, 1997, a spectrum Φ(E) of the radiation 101 is expressed by Expression (3) and Expression (4).

$$\begin{matrix}Φ ( E ) = {\sum }_{i = 0}^{n} {a}_{i} ( E ) {V}^{i} ( E ≦ k V ) & ( 3 ) \\ Φ ( E ) = 0 ( otherwise ) & ( 4 )\end{matrix}$$

In Expression (3), aᵢ(E) represents a coefficient determined by measuring the spectrum, and V represents a set tube voltage of the radiation tube bulb (Vⁱ represents V raised to the i-th power). In Expression (4), “otherwise” indicates a range other than the previously defined range (in the case of Expression (3) and Expression (4), a range other than E≤kV).

Therefore, assuming that the tube voltage waveform obtained in Step S402 is represented by V(t), the spectrum at a time “t” is expressed by Expression (5) and Expression (6).

$$\begin{matrix}Φ ( E ( t ) ) = {\sum }_{i = 0}^{n} {{a}_{i} ( E ) [ V ( t ) ]}^{i} ( E ≦ k V ) & ( 5 ) \\ Φ ( E ( t ) ) = 0 ( otherwise ) & ( 6 )\end{matrix}$$

The spectrum of the low-energy radiation 101 corresponding to the first tube voltage V₁ is expressed by Expression (7).

$$\begin{matrix}{Φ}_{Low} ( E ) = {\int }_{{t}_{2}}^{{t}_{3}} Φ [ E ( t ) ] dt \\ = \{ \begin{matrix}{\int }_{{t}_{2}}^{{t}_{3}} {\sum }_{i = 0}^{n} {{a}_{i} ( E ) [ V ( t ) ]}^{i} dt ( E ≦ k V ) \\ 0 ( otherwise )\end{matrix}\end{matrix} \qquad ( 7 )$$

In Expression (7), t₂ represents a radiation application start time of the first tube voltage V₁, and t₃ represents a radiation application end time of the first tube voltage V₁ as shown in FIG. 3B.

The spectrum of the high-energy radiation 101 corresponding to the second tube voltage V₂ is expressed by Expression (8).

$$\begin{matrix}{Φ}_{High} ( E ) = {\int }_{{t}_{3}}^{{t}_{5}} Φ [ E ( t ) ] dt \\ = \{ \begin{matrix}{\int }_{{t}_{3}}^{{t}_{5}} {\sum }_{i = 0}^{n} {{a}_{i} ( E ) [ V ( t ) ]}^{i} dt ( E ≦ k V ) \\ 0 ( otherwise )\end{matrix}\end{matrix} \qquad ( 8 )$$

In Expression (8), t₃ represents a radiation application start time of the second tube voltage V₂, and is represents a radiation application end time of the second tube voltage V₂ as shown in FIG. 3B. The time t₂, the time t₃, and the time t₅ are equal to the timings of the sampling and holding, and hence the spectrum calculating unit 132 can determine the spectrum from the timings of the sampling and holding.

The time t₃ and the time t₅ are changed depending on the pulse width of the radiation 101 set by the operator or the pulse width calculated by the AEC processing unit 134. Therefore, when those changes are performed, the spectrum calculating unit 132 again determines the spectrum based on the changed pulse width. Expression (7) and Expression (8) may also be standardized by their respective integration ranges as the requirement arises.

The spectrum calculating unit 132 further performs processing for appropriately correcting the thus calculated spectrum of the radiation 101 in consideration of various factors for determining the spectrum, which include the inherent filtration of the radiation tube bulb, the presence or absence of the radiation filter 102, the presence or absence of the stand 103 (positional information including an angle), and a front cover of the radiation detector 120. Specifically, the spectrum calculating unit 132 determines, for example, the (exponentially attenuating) energy-specific attenuation amount of the number of photons at the time of transmission through each substance, based on the thickness of each substance and an energy-dependent attenuation coefficient in the radiation detector 120, and performs multiplication to correct the spectrum.

Subsequently, in Step S404, the spectral-spatial information calculating unit 133 calculates spectral-spatial information through use of the spectrum of the radiation 101 calculated in Step S403. As described above, examples of the spectral-spatial information calculated in this case include the spatial distribution of the thickness of each substance and the spatial distribution of the effective atomic number and the areal density. When the processing of Step S404 ends, the processing of the flow chart illustrated in FIG. 4 is brought to an end.

Next, examples of a specific method of acquiring the tube voltage waveform of the radiation generator 110 (acquisition method illustrated in Step S402 in FIG. 4) in the first embodiment are described.

[First Acquisition Example of Acquiring Tube Voltage Waveform]

A first acquisition example of acquiring the tube voltage waveform of the radiation generator 110 is a mode of actually measuring the transient response characteristic of the radiation tube bulb being the radiation generator 110 through use of the voltmeter 113 or another such measuring device provided to the radiation imaging apparatus 100.

FIG. 5 is a diagram for illustrating an example of a functional configuration of the spectrum calculating unit 132 relating to the first acquisition example of acquiring the tube voltage waveform of the radiation generator 110 illustrated in FIG. 1A in the first embodiment of the present invention. In this case, the spectrum calculating unit 132 illustrated in FIG. 5 is referred to as “spectrum calculating unit 132-11”.

The spectrum calculating unit 132-11 illustrated in FIG. 5 includes functional components of a waveform acquiring unit 510, a tube voltage deriving unit 520, and a spectrum conversion unit 530. The tube voltage deriving unit 520 includes a tube bulb information storing unit 521 and a waveform processing unit 522.

The waveform acquiring unit 510 acquires the tube voltage waveform of the radiation generator 110 from, for example, the voltmeter 113 provided to the radiation imaging apparatus 100. The waveform acquiring unit 510 can also employ a form of acquiring the tube voltage waveform of the radiation generator 110 through calculation based on information of a radiation dosimeter included in the measuring device 160.

The tube voltage deriving unit 520 derives the voltage waveform of the radiation generator 110 based on the transient response characteristic of the radiation generator 110 stored in the tube bulb information storing unit 521 and a voltage waveform acquired by the waveform acquiring unit 510. Specifically, the tube bulb information storing unit 521 stores information on the transient response characteristic of the radiation generator 110. The waveform processing unit 522 processes the voltage waveform acquired by the waveform acquiring unit 510 through use of the transient response characteristic of the radiation generator 110 stored in the tube bulb information storing unit 521, to thereby derive the voltage waveform of the radiation generator 110.

The spectrum conversion unit 530 calculates the spectrum of the radiation 101 through conversion based on the voltage waveform of the radiation generator 110 derived by the tube voltage deriving unit 520.

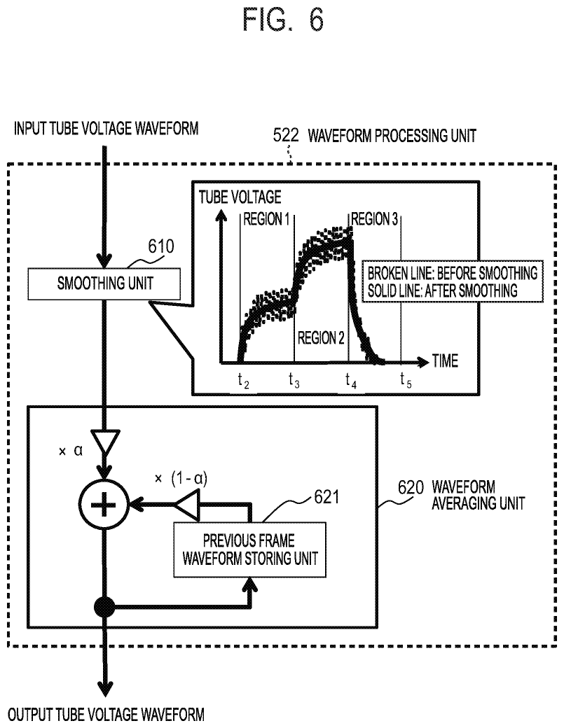

FIG. 6 is a diagram for illustrating an example of a functional configuration of the waveform processing unit 522 illustrated in FIG. 5. The waveform processing unit 522 illustrated in FIG. 5 includes a smoothing unit 610 and a waveform averaging unit 620 as illustrated in FIG. 6.

The smoothing unit 610 performs smoothing processing for smoothing the tube voltage waveform acquired by the waveform acquiring unit 510 (described as “input tube voltage waveform” in FIG. 6). Specifically, the smoothing unit 610 performs the smoothing processing for each pulse of the radiation 101 (for each frame in terms of an acquired radiation image). Examples of the method of smoothing to be performed by the smoothing unit 610 include a moving average or a polynomial fitting and an exponential function fitting that use a method of least squares. At this time, the fitting can be performed by, for example, dividing the tube voltage waveform acquired by the waveform acquiring unit 510 into a region of the first tube voltage V₁ (region 1), a region of the second tube voltage V₂ (region 2), and a region of a wave tail (region 3). In addition, for example, when the moving average is derived, the processing is performed so as to exclude the other regions for the derivation of the moving average. Further, for example, when the polynomial fitting is performed, different coefficients are used for respective regions.

The waveform averaging unit 620 performs processing for averaging a plurality of tube voltage waveforms processed by the smoothing unit 610 with respect to a plurality of tube voltage waveforms acquired in time series by the waveform acquiring unit 510. Then, the waveform averaging unit 620 outputs a tube voltage waveform obtained by the averaging processing (described as “output tube voltage waveform” in FIG. 6). Specifically, the waveform averaging unit 620 performs the averaging processing among a plurality of pulses of the radiation 101 (among a plurality of frames in terms of an acquired radiation image). That is, the waveform averaging unit 620 performs the averaging processing in a time direction for the plurality of tube voltage waveforms processed by the smoothing unit 610. The averaging processing in the time direction can be performed recursively in accordance with, for example, Expression (9).

(output tube voltage waveform)=α×(observed waveform subjected to smoothing)+(1−α)×(previous frame waveform) (9)

In Expression (9), a represents a coefficient. The tube voltage waveform obtained by the averaging processing of the waveform averaging unit 620 (described as “output tube voltage waveform” in FIG. 6) is stored in a previous frame waveform storing unit 621, and is used to calculate the output tube voltage waveform of the next frame. At this time, information of several past frames may be simply averaged.

There is also a case in which an appropriate tube voltage waveform can be obtained by setting an appropriate initial value even when the number of pulses of the radiation 101 is small. The initial value for this case is switched every time a transition in mode is caused, and is stored in the previous frame waveform storing unit 621 before the first radiation application is started. As an example of the initial value for this case, a tube voltage waveform to be input (that is, the waveform at a time when the impulse response is assumed to be a delta function) may be used as it is. For example, such a step function as expressed by Expression (10) is used.

$$V ( t ) = \{ \begin{matrix}0 otherwise \\ {V}_{1} ( {t}_{2} ≦ t ≦ {ι}_{3} ) \\ {V}_{2} ( {ι}_{3} ≦ t ≦ {t}_{4} )\end{matrix} \qquad ( 10 )$$

In Expression (10), t₂ represents an X-ray application start time of the first tube voltage V₁, t₃ represents an X-ray application end time of the first tube voltage V₁, and t₄ represents a time at which the maximum tube voltage of the second tube voltage V₂ is reached as shown in FIG. 3B. Expression (10) indicates a state in which there is no transient response of the radiation tube bulb being the radiation generator 110. Through the repetitive measurement, such a transient response characteristic as shown in FIG. 3B is gradually approached due to the effect of a recursive filter, and the transient response of the radiation tube bulb can be obtained.

When changes in tube voltage waveform in other ranges before and after the mode transition are small, the tube voltage waveform before the mode transition may be used as the initial value as it is. As another initial value, a method of storing an expected tube voltage waveform in the tube bulb information storing unit 521 in advance can be used. When the expected tube voltage waveform is to be calculated as the initial value, any one of a second acquisition example, a third acquisition example, and a fourth acquisition example of acquiring a tube voltage waveform, which are described later, may be used (those acquisition examples each use a calculated value, and are used in combination with the first acquisition example of acquiring the tube voltage waveform obtained from the actually measured value, to thereby improve the accuracy of the tube voltage waveform).

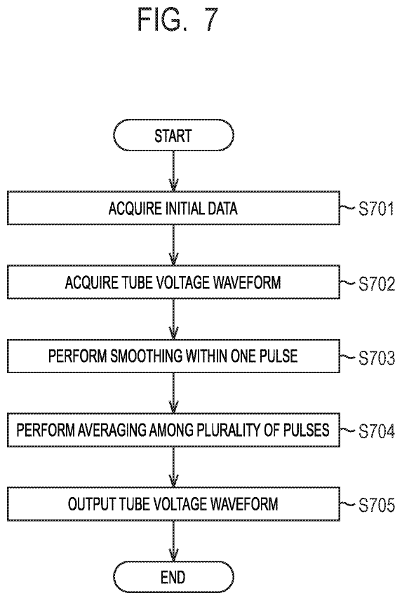

FIG. 7 is a flow chart for illustrating an example of a processing procedure of the waveform processing unit 522 illustrated in FIG. 6.

First, in Step S701, the waveform averaging unit 620 stores the above-mentioned initial value of the tube voltage waveform in the previous frame waveform storing unit 621.

Subsequently, in Step S702, the smoothing unit 610 acquires the tube voltage waveform (described as “input tube voltage waveform” in FIG. 6) from the waveform acquiring unit 510.

Subsequently, in Step S703, the smoothing unit 610 performs processing for smoothing the tube voltage waveform acquired in Step S702 for each pulse of the radiation 101 (each frame in terms of an acquired radiation image).

Subsequently, in Step S704, the waveform averaging unit 620 performs processing for averaging the plurality of tube voltage waveforms processed by the smoothing unit 610 among a plurality of pulses of the radiation 101 (among a plurality of frames in terms of an acquired radiation image). The waveform averaging unit 620 performs this averaging processing recursively.

Subsequently, in Step S705, the waveform averaging unit 620 outputs the tube voltage waveform obtained by the averaging processing (described as “output tube voltage waveform” in FIG. 6). Then, when the processing of Step S705 ends, the processing of the flow chart illustrated in FIG. 7 is brought to an end.

In the first acquisition example of acquiring the tube voltage waveform, which is illustrated in FIG. 5 to FIG. 7, it is possible to prepare the plausible spectrum of the radiation 101 in advance before the imaging even for the first acquired image by setting the initial value of the tube voltage waveform. With this, the real-time property can be significantly improved. Further, through provision of the smoothing unit 610 and the waveform averaging unit 620, it is possible to improve the measurement accuracy of the tube voltage waveform, and to therefore calculate a highly accurate spectrum of the radiation 101. In addition, through application of the recursive filter to the averaging processing in the time direction, it is possible to continuously shift from the first determined initial value to the spectrum of the radiation 101 based on the actually measured value, and to greatly reduce a sense of discomfort due to discontinuous changes.

[Second Acquisition Example of Acquiring Tube Voltage Waveform]

The second acquisition example of acquiring the tube voltage waveform of the radiation generator 110 is a mode of holding measurement results of the tube voltage under various imaging conditions in advance, and obtaining the tube voltage waveform under set imaging conditions through interpolation or extrapolation.

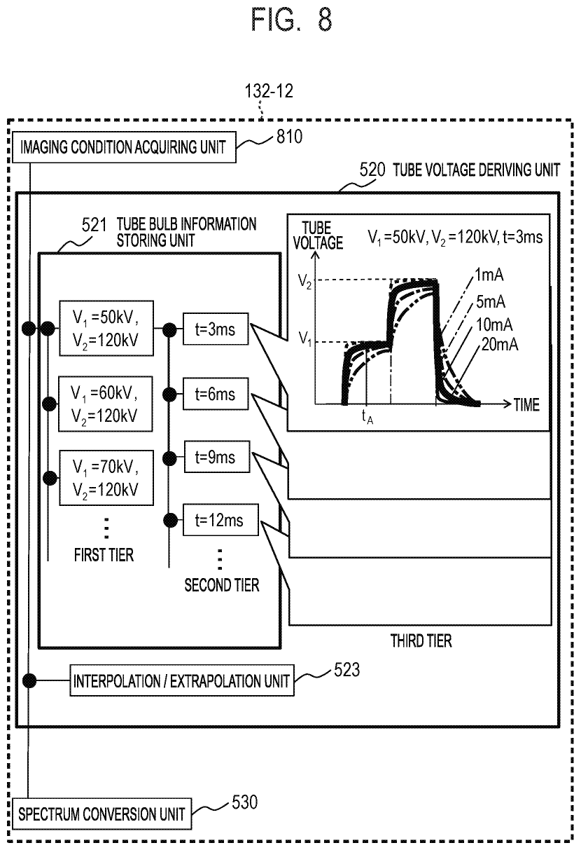

FIG. 8 is a diagram for illustrating an example of a functional configuration of the spectrum calculating unit 132 relating to the second acquisition example of acquiring the tube voltage waveform of the radiation generator 110 illustrated in FIG. 1A in the first embodiment of the present invention. In this case, the spectrum calculating unit 132 illustrated in FIG. 8 is referred to as “spectrum calculating unit 132-12”. In FIG. 8, the same components as those illustrated in FIG. 5 are denoted by the same reference symbols, and detailed descriptions thereof are omitted.

The spectrum calculating unit 132-12 illustrated in FIG. 8 includes functional components of an imaging condition acquiring unit 810, the tube voltage deriving unit 520, and the spectrum conversion unit 530. The tube voltage deriving unit 520 includes the tube bulb information storing unit 521 and an interpolation/extrapolation unit 523.

The imaging condition acquiring unit 810 acquires imaging conditions relating to radiation imaging of the radiation generator 110.

The tube voltage deriving unit 520 derives the voltage waveform of the radiation generator 110 based on the transient response characteristic of the radiation generator 110 stored in the tube bulb information storing unit 521 and the imaging conditions acquired by the imaging condition acquiring unit 810. Specifically, the tube bulb information storing unit 521 stores information on the transient response characteristic of the radiation generator 110. In the second acquisition example of acquiring the tube voltage waveform, the tube bulb information storing unit 521 stores the tube voltage waveform acquired in advance. The tube voltage waveform is acquired through use of the voltmeter 113 or through use of the radiation dosimeter provided to the measuring device 160 at the time of calibration. In the second acquisition example of acquiring the tube voltage waveform, the voltmeter 113 and the radiation dosimeter are required only at the time of calibration, and may therefore be formed so as to be removable. The interpolation/extrapolation unit 523 derives the tube voltage waveform of the radiation generator 110 by performing the interpolation or extrapolation based on the imaging conditions acquired by the imaging condition acquiring unit 810 through use of the transient response characteristic of the radiation generator 110 stored in the tube bulb information storing unit 521.

The spectrum conversion unit 530 calculates the spectrum of the radiation 101 through conversion based on the voltage waveform of the radiation generator 110 derived by the tube voltage deriving unit 520.

In the second acquisition example of acquiring the tube voltage waveform, the tube voltage set by the operator at the time of mode transition is referred to as “set tube voltage”, and the tube voltage acquired through use of the voltmeter 113 or the radiation dosimeter is referred to as “observed tube voltage”. The tube current set by the operator is referred to as “set tube current”, and the pulse width set by the operator is referred to as “set pulse width”. The observed tube voltage waveform means the waveform of the observed tube voltage.

An observed tube voltage waveform is stored in association with acquisition conditions at the time of calibration (calibration conditions). Items of the calibration conditions include the set tube voltage, the set tube current, and the set pulse width. The interpolation/extrapolation unit 523 derives the observed tube voltage waveform under the imaging conditions set by interpolating or extrapolating the observed tube voltage waveform acquired in advance based on the imaging conditions set at the time of mode transition.

Now, a data structure and an interpolation method of the tube bulb information storing unit 521 illustrated in FIG. 8 are described. Information on the observed tube voltage waveform is organized in a tree structure as illustrated in the tube bulb information storing unit 521 illustrated in FIG. 8. In the second acquisition example of acquiring the tube voltage waveform, the first tier classifies the information by the set tube voltage, the second tier classifies the information by the set pulse width, and the third tier classifies the information by the set tube current. The interpolation/extrapolation unit 523 first performs interpolation on the set tube current in the third tier, then performs interpolation on the set pulse width in the second tier, and lastly performs interpolation on the set tube voltage in the first tier.

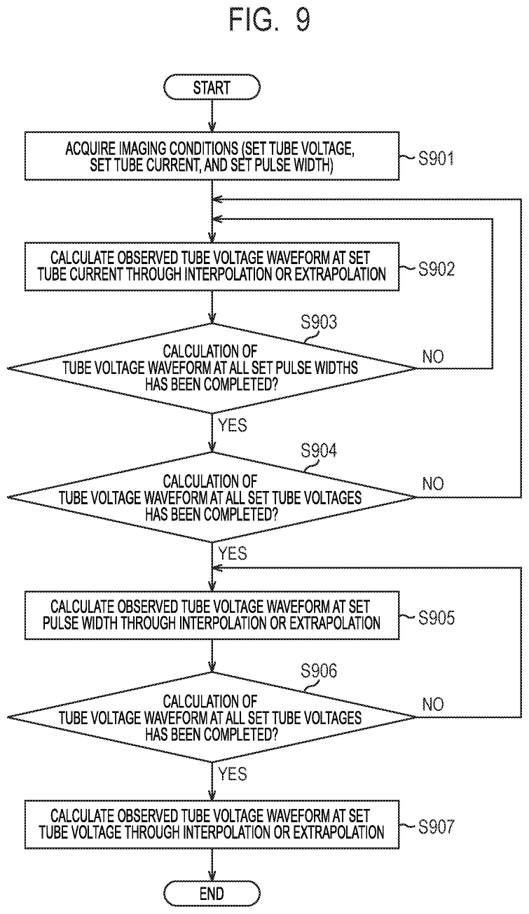

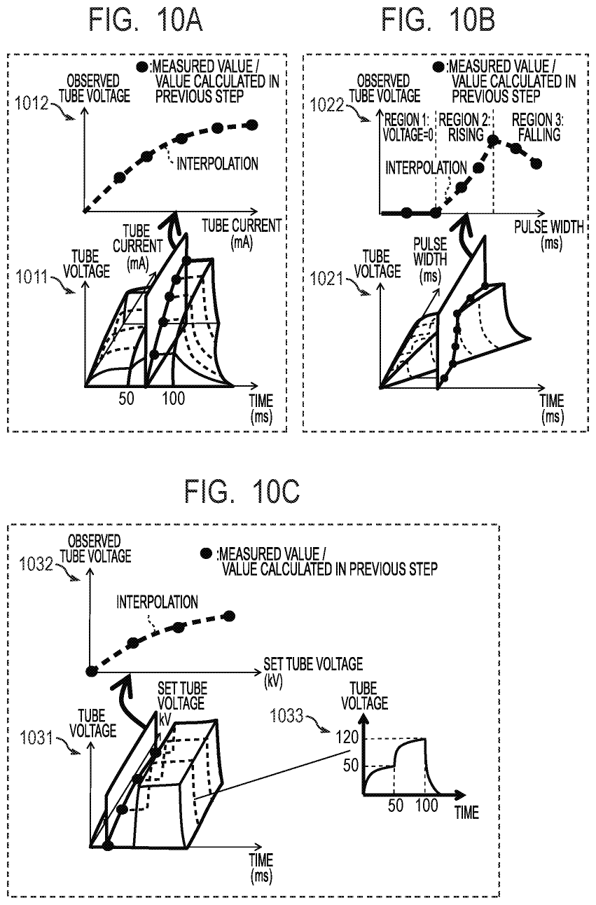

FIG. 9 is a flow chart for illustrating an example of a processing procedure of the imaging condition acquiring unit 810 and the tube voltage deriving unit 520 illustrated in FIG. 8. FIG. 10A to FIG. 10C are explanatory graphs for showing processing of the interpolation/extrapolation unit 523 shown in FIG. 8.

First, in Step S901, the imaging condition acquiring unit 810 acquires the imaging conditions set at the time of mode transition. In the second acquisition example of acquiring the tube voltage waveform, the imaging conditions to be acquired in this case are set as the set tube voltage, the set tube current, and the set pulse width, but the present invention is not limited to this form. For example, information on the temperature and the heat unit of the radiation tube bulb may be acquired as the imaging conditions. In this case, an interpolation process for the temperature or the heat unit is inserted between Step S901 and Step S902.

Subsequently, in Step S902, the interpolation/extrapolation unit 523 calculates the observed tube voltage waveform at the set tube current in the third tier described above through interpolation or extrapolation. FIG. 10A shows a concept of the processing of Step S902 (specifically, the interpolation processing of the interpolation/extrapolation unit 523). Specifically, the interpolation/extrapolation unit 523 obtains through interpolation a relationship between the tube current and the observed tube voltage, which is shown in a display area 1012, for each time of the observed tube voltage waveform shown in a display area 1011, and derives the values of the observed tube voltages with respect to the set tube currents for each time. As the interpolation performed at this time, a general method, for example, the polynomial fitting based on the method of least squares is used.

Subsequently, in Step S903, the interpolation/extrapolation unit 523 determines whether or not the calculation of the observed tube voltage waveform at all the set pulse widths has been completed. When determining as a result of this determination that the calculation of the observed tube voltage waveform at all the set pulse widths has not been completed yet (N in Step S903), the interpolation/extrapolation unit 523 returns to Step S902 to again perform the processing of Step S902.

Meanwhile, when determining as a result of the determination of Step S903 that the calculation of the observed tube voltage waveform at all the set pulse widths has been completed (Y in Step S903), the interpolation/extrapolation unit 523 advances to Step S904. In Step S904, the interpolation/extrapolation unit 523 determines whether or not the calculation of the observed tube voltage waveform at all the set tube voltages has been completed. When determining as a result of this determination that the calculation of the observed tube voltage waveform at all the set tube voltages has not been completed yet (N in Step S904), the interpolation/extrapolation unit 523 returns to Step S902 to again perform Step S902 and the subsequent processing steps.

Meanwhile, when determining as a result of the determination of Step S904 that the calculation of the observed tube voltage waveform at all the set tube voltages has been completed (Y in Step S904), the interpolation/extrapolation unit 523 advances to Step S905. In Step S905, the interpolation/extrapolation unit 523 calculates the observed tube voltage waveform at the set pulse width in the second tier described above through interpolation or extrapolation. FIG. 10B shows a concept of the processing of Step S905 (specifically, the interpolation processing of the interpolation/extrapolation unit 523). Specifically, the interpolation/extrapolation unit 523 obtains through interpolation a relationship between the pulse width and the observed tube voltage, which is shown in a display area 1022 for each time of the observed tube voltage waveform shown in a display area 1021, and derives the values of the observed tube voltages with respect to the set pulse widths for each time. At this time of interpolation, it is possible to derive different expressions by dividing the observed tube voltage waveform into the region 1 in which the voltage is 0, the region 2 being a rising region, and the region 3 being a falling region.

Subsequently, in Step S906, the interpolation/extrapolation unit 523 determines whether or not the calculation of the observed tube voltage waveform at all the set tube voltages has been completed. When determining as a result of this determination that the calculation of the observed tube voltage waveform at all the set tube voltages has not been completed yet (N in Step S906), the interpolation/extrapolation unit 523 returns to Step S905 to again perform the processing of Step S905.

Meanwhile, when determining as a result of the determination of Step S906 that the calculation of the observed tube voltage waveform at all the set tube voltages has been completed (Y in Step S906), the interpolation/extrapolation unit 523 advances to Step S907. In Step S907, the interpolation/extrapolation unit 523 calculates the observed tube voltage waveform at the set tube voltages in the first tier described above through interpolation or extrapolation. FIG. 10C shows a concept of the processing of Step S907 (specifically, the interpolation processing of the interpolation/extrapolation unit 523). Specifically, the interpolation/extrapolation unit 523 obtains through interpolation a relationship between the set tube voltages and the observed tube voltage, which is shown in a display area 1032 for each time of the observed tube voltage waveform (including the observed tube voltage waveform shown in a display area 1033) shown in a display area 1031, and derives the values of the observed tube voltages with respect to the set tube voltages for each time. When the processing of Step S907 ends, the processing of the flow chart illustrated in FIG. 9 is brought to an end.

When emphasis is placed on the accuracy of interpolation performed by the interpolation/extrapolation unit 523, it is desired to perform processing for all combinations of the set tube voltage and the set tube current as in the example illustrated in FIG. 9. However, when emphasis is not placed on the accuracy of interpolation, the interpolation of some data can be omitted. For example, when the tube voltage waveforms relating to the imaging conditions are stored in the tube bulb information storing unit 521 illustrated in FIG. 8 and conditions described as “set conditions” shown in Table 1 are set by the mode transition, it suffices that the tube voltage waveforms to be used for the interpolation are only the tube voltage waveforms under four interpolation conditions [1] to [4] shown in Table 1, which are calibration conditions adjacent to the imaging conditions.

TABLE 1

| Set | Interpolation | Interpolation | Interpolation | Interpolation |
|---|---|---|---|---|
| conditions | condition [1] | condition [2] | condition [3] | condition [4] |
| V1 = 55 kV | V1 = 50 kV | V1 = 50 kV | V1 = 60 kV | V1 = 60 kV |
| V2 = 120 kV | V2 = 120 kV | V2 = 120 kV | V2 = 120 kV | V2 = 120 kV |
| t = 5 ms | t = 3 ms | t = 6 ms | t = 3 ms | t = 6 ms |
| I = 3 mA | | | | |

V1: First tube voltage,
V2: Second tube voltage,
t: Pulse width,
I: Tube current

In this case, the interpolation/extrapolation unit 523 first obtains a tube voltage waveform at a tube current I=3 mA under each of the four interpolation conditions [1] to [4] shown in Table 1. After that, the interpolation/extrapolation unit 523 derives a tube current waveform at (V₁, V₂, t, I)=(50 kV, 120 kV, 5 ms, 3 mA) using the interpolation condition [1] and the interpolation condition [2]. The interpolation/extrapolation unit 523 further derives a tube current waveform at (V₁, V₂, t, I)=(60 kV, 120 kV, 5 ms, 3 mA) using the interpolation condition [3] and the interpolation condition [4]. Then, the interpolation/extrapolation unit 523 again interpolates those two tube voltage waveforms to derive a tube voltage waveform under the set conditions.

In the above-mentioned example, the calibration is performed before the radiation imaging, but may be performed after the radiation imaging as well. In general, the calibration is performed at a time other than the time of radiation application for image formation.

[Third Acquisition Example of Acquiring Tube Voltage Waveform]

In the second acquisition example of acquiring the tube voltage waveform described above, there is a concern that the information on the held (observed) tube voltage waveforms may become enormous. In view of this, the third acquisition example of acquiring the tube voltage waveform is a mode of, on the assumption that the radiation tube bulb being the radiation generator 110 is a linear and time-invariant (LTI) system, holding the impulse response of the radiation tube bulb instead of holding the tube voltage waveform, and acquiring the tube voltage waveform through use of the impulse response.

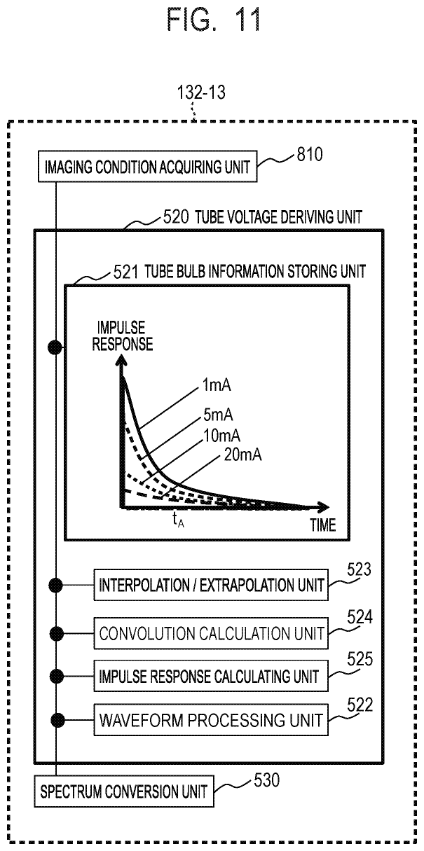

FIG. 11 is a diagram for illustrating an example of a functional configuration of the spectrum calculating unit 132 relating to the third acquisition example of acquiring the tube voltage waveform of the radiation generator 110 illustrated in FIG. 1A in the first embodiment of the present invention. In this case, the spectrum calculating unit 132 illustrated in FIG. 11 is referred to as “spectrum calculating unit 132-13”. In FIG. 11, the same components as those illustrated in FIG. 5 and FIG. 8 are denoted by the same reference symbols, and detailed descriptions thereof are omitted.

The spectrum calculating unit 132-13 illustrated in FIG. 11 includes functional components of the imaging condition acquiring unit 810, the tube voltage deriving unit 520, and the spectrum conversion unit 530. The tube voltage deriving unit 520 includes the tube bulb information storing unit 521, the waveform processing unit 522, the interpolation/extrapolation unit 523, a convolution calculation unit 524, and an impulse response calculating unit 525.

The tube bulb information storing unit 521 stores information on an impulse response characteristic as the transient response characteristic of the radiation generator 110. Specifically, in the third acquisition example of acquiring the tube voltage waveform, the impulse response is stored in the tube bulb information storing unit 521 in association with impulse response acquisition conditions at the time of calibration (calibration conditions). In this case, items of the calibration conditions include the set tube voltage, the set tube current, and the set pulse width. In the example illustrated in FIG. 11, the impulse response at each tube current is stored in the tube bulb information storing unit 521.

A convolution calculation unit 524 performs convolution processing relating to the impulse response. An impulse response calculating unit 525 performs calculation relating to the impulse response.

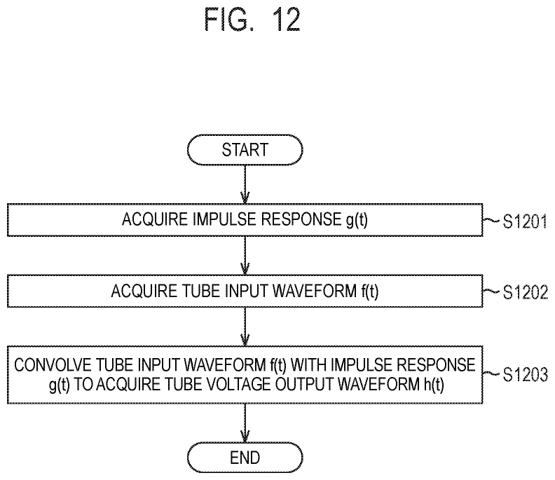

FIG. 12 is a flow chart for illustrating an example of a processing procedure of the tube voltage deriving unit 520 illustrated in FIG. 11.

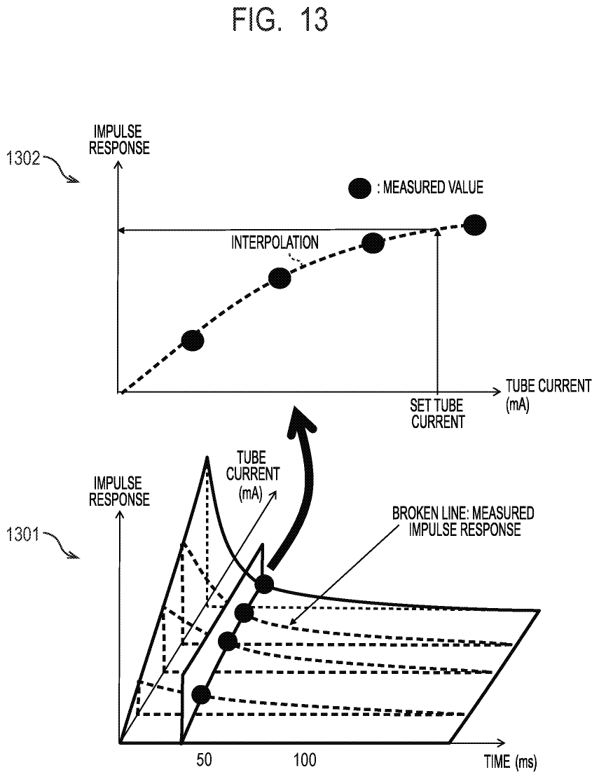

First, in Step S1201, the impulse response calculating unit 525 calculates an impulse response g(t). For the calculation of the impulse response g(t), information on a transition mode and a change state, which is acquired by the imaging condition acquiring unit 810, is used. In addition, the impulse response has, for example, tube current dependency, radiation tube bulb temperature dependency, and radiation tube bulb heat unit dependency, and hence it is required to appropriately calculate impulse responses corresponding to the information acquired by the imaging condition acquiring unit 810. As an example, a calculation method performed when the tube current of the radiation tube bulb being the radiation generator 110 changes is described below with reference to FIG. 13.

FIG. 13 is explanatory graphs for showing an example of calculating an impulse response by the impulse response calculating unit 525 illustrated in FIG. 11. In this case, the tube bulb information storing unit 521 stores in advance a plurality of measurement results of the impulse response of the radiation tube bulb associated with the tube current. In addition, in the spectrum calculating unit 132-13 illustrated in FIG. 11, the impulse response at the set tube current shown in a display area 1302 is obtained by interpolating or extrapolating the measurement results of the impulse response for each time by the interpolation/extrapolation unit 523 and the impulse response calculating unit 525 at each time. In a display area 1301, impulse responses at a plurality of measured tube currents are arranged in a relationship among the time, the tube current, and the impulse response.

Further, the impulse response is acquired in advance at the time of calibration. For example, the impulse response can be calculated by the impulse response calculating unit 525 in exactly the same manner for the temperature and the heat unit of the radiation tube bulb. That is, in this case, the temperature and the heat unit of the radiation tube bulb may be stored in the tube bulb information storing unit 521 in association with the impulse response of the radiation tube bulb, and may be interpolated in the same manner as in the case of the tube current.

Now, the description returns to FIG. 12 again. Subsequently, in Step S1202, the tube voltage deriving unit 520 acquires a tube bulb input waveform f(t) relating to the tube voltage waveform of the radiation generator 110. For example, the step function described above in Expression (8) is used for the tube voltage waveform in this case, but the first embodiment is not limited thereto. Particularly in the third acquisition example of acquiring the tube voltage waveform, the greatest advantage is that the spectrum of the radiation 101 can be obtained for the input of any waveform (for example, a triangular wave) other than the step function described above in Expression (10).

Subsequently, in Step S1203, for example, the convolution calculation unit 524 uses the impulse response g(t) acquired in Step S1201 and the tube bulb input waveform f(t) acquired in Step S1202 to calculate a tube voltage output waveform h(t) through convolution. Specifically, a tube voltage output h(t) is calculated by Expression (11).

h(t)=∫_{-∞}^{∞}f(s)g(t−s)ds (11)

After the tube voltage output waveform h(t) is successfully acquired in Step S1203, the spectrum of the radiation 101 is calculated in accordance with, for example, the processing of the flow chart illustrated in FIG. 4.

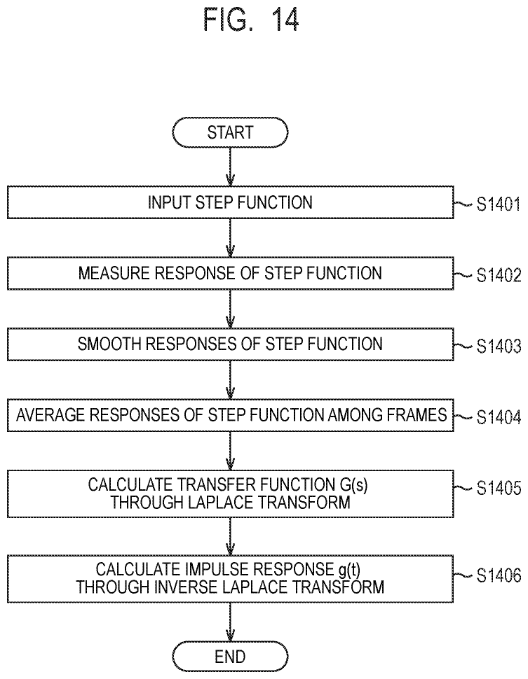

FIG. 14 is a flow chart for illustrating an example of a processing procedure in an impulse response calibration method relating to the third acquisition example of acquiring the tube voltage waveform of the radiation generator 110 illustrated in FIG. 1A in the first embodiment of the present invention. The calibration in this example can be achieved by, for example, the impulse response calculating unit 525 performing a Laplace transform or an inverse Laplace transform on a step response.

First, in Step S1401, for example, the impulse response calculating unit 525 inputs a step function. Examples of the function input in this case include a one-step function shown in Expression (12).

$$V ( t ) = \{ \begin{matrix}0 ( t < {t}_{A} ) \\ {V}_{s} ( {t}_{A} ≦ t ≦ {t}_{B} ) \\ 0 ( {t}_{B} < t )\end{matrix} \qquad ( 12 )$$

In Expression (12), there may be used such a step function that can assume that t_{A} is zero and t_{B} is infinity, thereby involving no fall.

Subsequently, in Step S1402, for example, the impulse response calculating unit 525 measures the response (tube voltage waveform) of the step function input in Step S1401. The measurement is performed through use of, for example, the voltmeter 113 or the radiation dosimeter provided to the measuring device 160. After the calibration is completed and a set of impulse responses is stored in the tube bulb information storing unit 521, the voltmeter 113 and the radiation dosimeter may be removed.

Subsequently, in Step S1403, the waveform processing unit 522 (smoothing unit 610) smoothes the responses of the step function obtained in Step S1402 within one pulse.

Subsequently, in Step S1404, the waveform processing unit 522 (waveform averaging unit 620) performs processing for averaging results obtained in Step S1403 among frames.

Subsequently, in Step S1405, the impulse response calculating unit 525 performs a Laplace transform on the response of the step function processed in Step S1403 and Step S1404 to calculate a transfer function. In this case, assuming that the Laplace transform of the input (calculated numerically from the input) is represented by F(s), the transfer function is represented by G(s), and the Laplace transform of the output is represented by H(s), Expression (13) is obtained.

H(s)=G(s)F(s) (13)

The Laplace transform of the step function becomes F(s)=1/s, and hence a transfer function G(s) becomes Expression (14).

$$G ( s ) = \frac{H ( s )}{F ( s )} = sH ( s ) \qquad ( 14 )$$

Subsequently, in Step S1406, the impulse response calculating unit 525 obtains the impulse response g(t) by performing an inverse Laplace transform on the transfer function G(s) obtained in Step S1405. When the processing of Step S1406 ends, the processing of the flow chart illustrated in FIG. 14 is brought to an end.

In the third acquisition example of acquiring the tube voltage waveform, the voltmeter 113 and the radiation dosimeter provided to the measuring device 160 are required only at the time of calibration, and may therefore be formed so as to be removable. Further, in the third acquisition example of acquiring the tube voltage waveform, the step function is input to obtain the impulse response of the radiation tube bulb from its response. Instead, an impulse function may be input to directly obtain the impulse response by performing a Fourier transform or an inverse Fourier transform. In addition, for the confirmation of the linearity and time-invariance of the radiation tube bulb, an impulse response is obtained under certain conditions by the above-mentioned method to compare actually measured tube voltage waveforms, which are obtained when the tube current is fixed and the set tube voltage and pulse width are changed in various manners, with tube voltage waveform calculated from the obtained impulse response. If the linearity and time-invariance are not achieved with high accuracy, it is required to appropriately modify the impulse response based on the input value.

In the above-mentioned example, the transfer function is obtained through use of the Laplace transform, but may be obtained through use of a z-transform or another such similar method. Further, if the parameter dependency (for example, tube current dependency, radiation tube bulb temperature dependency, and radiation tube bulb heat unit dependency) of the impulse response is negligible, such a process of interpolation illustrated in FIG. 13 can be omitted. Further, the calibration is performed before the radiation imaging, but may be performed after the radiation imaging. In general, the calibration is performed at a time other than the time of radiation application for image formation.

[Fourth Acquisition Example of Acquiring Tube Voltage Waveform]

In the fourth acquisition example of acquiring the tube voltage waveform, an output waveform is derived by obtaining an impulse response and convolving the impulse response with an input waveform in the same manner as in the third acquisition example of acquiring the tube voltage waveform described above. However, the fourth acquisition example of acquiring the tube voltage waveform is a mode of modeling the impulse response by a known function. This can significantly reduce the amount of calculation.

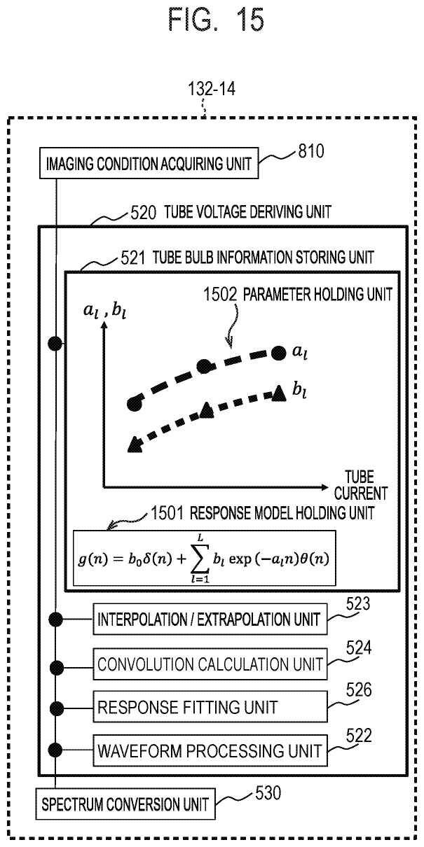

FIG. 15 is a diagram for illustrating an example of a functional configuration of the spectrum calculating unit 132 relating to the fourth acquisition example of acquiring the tube voltage waveform of the radiation generator 110 illustrated in FIG. 1A in the first embodiment of the present invention. In this case, the spectrum calculating unit 132 illustrated in FIG. 15 is referred to as “spectrum calculating unit 132-14”. In FIG. 15, the same components as those illustrated in FIG. 5, FIG. 8, and FIG. 11 are denoted by the same reference symbols, and detailed descriptions thereof are omitted.

The spectrum calculating unit 132-14 illustrated in FIG. 15 includes functional components of the imaging condition acquiring unit 810, the tube voltage deriving unit 520, and the spectrum conversion unit 530. The tube voltage deriving unit 520 includes the tube bulb information storing unit 521, the waveform processing unit 522, the interpolation/extrapolation unit 523, the convolution calculation unit 524, and a response fitting unit 526.

As illustrated in FIG. 15, the tube bulb information storing unit 521 includes a response model holding unit 1501 and a parameter holding unit 1502.

The response fitting unit 526 performs processing for fitting relating to a response model held by the response model holding unit 1501.

The response model relating to the impulse response of the radiation tube bulb, which is held by the response model holding unit 1501, can be expressed as, for example, a sum of a delta function and an exponential function having a plurality of (reciprocal) time constants as shown in Expression (15) and FIG. 15.

$$g ( n ) = {b}_{0} δ ( n ) + {\sum }_{l = 1}^{L} {b}_{l} exp ( - {a}_{l} n ) θ ( n ) \qquad ( 15 )$$

In Expression (15), “n” represents a discrete time, δ(n) represents a delta function (namely, δ(n)=1 (n=0) or 0 (n≠0)), exp(⋅) represents an exponential function, and θ(n) represents 0 (n<0) or 1 (n≥0).

In addition, b₀, a₁, and b₁ (l=1 to L) in Expression (15) are parameters that characterize the impulse response, and those values are stored in the parameter holding unit 1502. In addition, L in Expression (15) is the number of time constants, and may be set to, for example, 4.

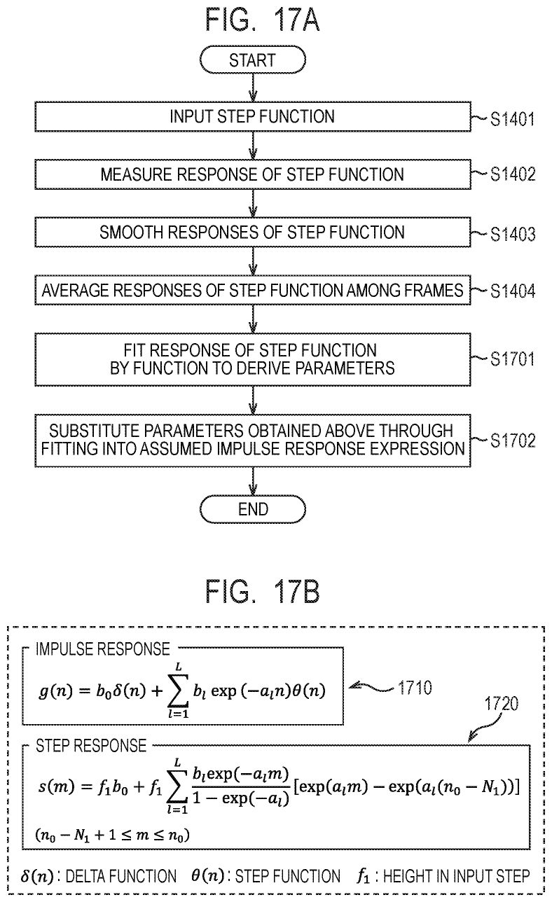

FIG. 17A is a flow chart for illustrating an example of a processing procedure in an impulse response calibration method relating to the fourth acquisition example of acquiring the tube voltage waveform of the radiation generator 110 illustrated in FIG. 1A in the first embodiment of the present invention. FIG. 17B shows Expression 1710 relating to the impulse response and Expression 1720 relating to the step response. In FIG. 17A, the same processing steps as those illustrated in FIG. 14 are denoted by the same step numbers.

First, in Step S1401 of FIG. 17A, for example, the response fitting unit 526 inputs a step function.

Subsequently, in Step S1402 of FIG. 17A, for example, the response fitting unit 526 measures the response (tube voltage waveform) of the step function input in Step S1401 of FIG. 17A.

Subsequently, in Step S1403 of FIG. 17A, the waveform processing unit 522 (smoothing unit 610) smoothes the responses of the step function obtained in Step S1402 of FIG. 17A within one pulse.

Subsequently, in Step S1404 of FIG. 17A, the waveform processing unit 522 (waveform averaging unit 620) performs processing for averaging results obtained in Step S1403 of FIG. 17A among frames.

Subsequently, in Step S1701 of FIG. 17A, the response fitting unit 526 subjects the response of the step function processed in Step S1403 and Step S1404 to the fitting with an analytical solution of the function, and derives the above-mentioned parameters.

Subsequently, in Step S1702 of FIG. 17A, the response fitting unit 526 obtains an impulse response by substituting the parameters derived in Step S1701 into an assumed impulse response expression. When the processing of Step S1702 ends, the processing of the flow chart illustrated in FIG. 17A is brought to an end.

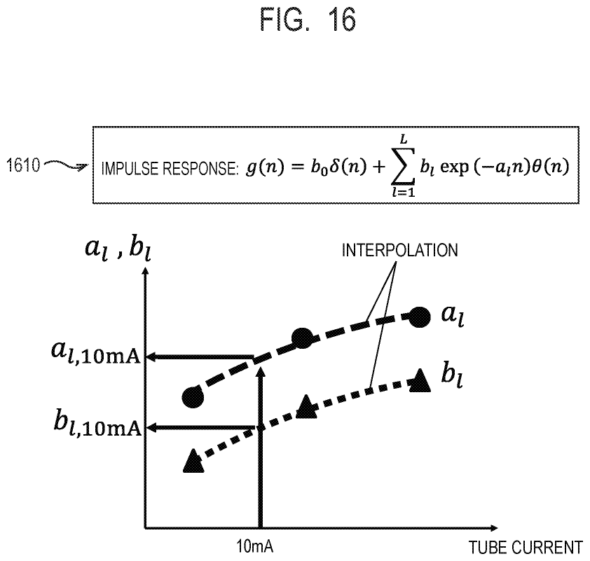

The impulse response has, for example, tube current dependency, radiation tube bulb temperature dependency, and radiation tube bulb heat unit dependency, and hence it is required to derive impulse responses corresponding to those values. In the fourth acquisition example of acquiring the tube voltage waveform, interpolation or extrapolation is performed on a parameter by the interpolation/extrapolation unit 523. The response fitting unit 526 obtains, for example, the parameter of the impulse response at each tube current, and fits the parameter to the graph having the horizontal axis representing the tube current and the vertical axis representing the parameter, which is shown in FIG. 16. Then, the response fitting unit 526 may input the parameter input at the time of the next mode transition to a fitting function, and may set the output value as a parameter to be used.

In the third acquisition example of acquiring the tube voltage waveform described above, it is required to hold data for all times and perform interpolation on the data. However, in the fourth acquisition example of acquiring the tube voltage waveform, only the parameters b₀, a₁, and b₁ (l=1 to L) are to be interpolated, with which the amount of calculation can be significantly reduced. For example, when L=4, nine pieces of data, namely, b₀, a₁, a₂, a₃, a₄, b₁, b₂, b₃, and b₄, may be interpolated.

For example, the impulse response can be calculated in exactly the same manner for the temperature and the heat unit of the radiation tube bulb as in the case of the third acquisition example of acquiring the tube voltage waveform described above.

For example, assuming the model of Expression (15) when the step function input in Step S1401 of FIG. 17A is represented by Expression (16), the step response can be analytically obtained as in Expression (17).

$$\begin{matrix} f ( n ) = \{ \begin{matrix}0 ( n < {n}_{0} - {N}_{1} + 1 ) \\ {f}_{1} ( {n}_{0} - {N}_{1} + 1 ≦ n ≦ {n}_{0} ) \\ 0 ( {n}_{0} + 1 ≦ n )\end{matrix} & ( 16 ) \\ s ( m ) = \{ \begin{matrix}0 ( m < {n}_{0} - {N}_{1} + 1 ) \\ {f}_{1} {b}_{0} + {f}_{1} {\sum }_{l = 1}^{L} \frac{{b}_{l}}{1 - {e}^{- {a}_{l}}} {e}^{- {a}_{l} m} ( {e}^{{a}_{l} m} - {e}^{{a}_{l} ( {n}_{0} - {N}_{1} )} ) ( {n}_{0} - {N}_{1} + 1 ≦ m ≦ {n}_{0} ) \\ {f}_{1} {\sum }_{l = 1}^{L} \frac{{b}_{l}}{1 - {e}^{- {a}_{l}}} {e}^{- {a}_{l} m} ( {e}^{{a}_{l} {n}_{0}} - {e}^{{a}_{l} ( {n}_{0} - {N}_{1} )} ) ( {n}_{0} + 1 ≦ m )\end{matrix} & ( 17 )\end{matrix}$$

In this case, in Step S1701 of FIG. 17A, the response fitting unit 526 fits Expression (17) to data on the time average of the responses of the step function obtained in Step S1404. The fitting may be performed through use of a generally used method by, for example, minimizing the square of a difference between the time average of the responses of the step function and Expression (17). Through the fitting, the parameters b₀, a₁, and b₁ are determined. Then, in Step S1702 of FIG. 17A, the response fitting unit 526 can obtain the impulse response by substituting the determined parameters into Expression (15).

The fourth acquisition example of acquiring the tube voltage waveform is advantageous in that, as described above, the number of pieces of data to be interpolated can be significantly reduced by modeling. The fourth acquisition example of acquiring the tube voltage waveform is also advantageous in that the output tube voltage waveform can be obtained not by a numerical solution but by an analytical solution (analytically obtained solution that can be expressed by a function or a coefficient). For example, when an input f(n) is such a step function as shown in Expression (18), its output h(m) (impulse response g(n) is expressed by Expression (15)) is expressed in a form shown in Expression (19).

$$\begin{matrix}f ( n ) = \{ \begin{matrix}0 otherwise \\ {f}_{1} ( {n}_{0} - {N}_{1} + 1 ≦ n ≦ {n}_{0} ) \\ {f}_{2} ( {n}_{0} + 1 ≦ n ≦ {n}_{0} + {N}_{2} )\end{matrix} & ( 18 ) \\ h ( m ) = \{ \begin{matrix}0 & ( m < {n}_{0} - {N}_{1} + 1 ) \\ \begin{matrix}{f}_{1} {b}_{0} + {f}_{1} {\sum }_{l = 1}^{L} \frac{{b}_{l}}{1 - {e}^{- {a}_{l}}} {e}^{- {a}_{l} m} \\ ( {e}^{{a}_{l} m} - {e}^{{a}_{l} ( {n}_{0} - {N}_{1} )} )\end{matrix} & ( {n}_{0} - {N}_{1} + 1 ≦ m ≦ {n}_{0} ) \\ \begin{matrix}{f}_{2} {b}_{0} + {f}_{1} {\sum }_{l = 1}^{L} \frac{{b}_{l}}{1 - {e}^{- {a}_{l}}} {e}^{- {a}_{l} m} \\ ( {e}^{{a}_{l} {n}_{0}} - {e}^{{a}_{l} ( {n}_{0} - {N}_{1} )} ) + \\ {f}_{2} {\sum }_{l = 1}^{L} \frac{{b}_{l}}{1 - {e}^{- {a}_{l}}} {e}^{- {a}_{l} m} ( {e}^{{a}_{l} m} - {e}^{{a}_{l} {n}_{0}} )\end{matrix} & ( {n}_{0} + 1 ≦ m ≦ {n}_{0} + {N}_{2} ) \\ \begin{matrix}{f}_{1} {\sum }_{l = 1}^{L} \frac{{b}_{l}}{1 - {e}^{- {a}_{l}}} {e}^{- {a}_{l} m} ( {e}^{{a}_{l} {n}_{0}} - {e}^{{a}_{l} ( {n}_{0} - {N}_{1} )} ) + \\ {f}_{2} {\sum }_{l = 1}^{L} \frac{{b}_{l}}{1 - {e}^{- {a}_{l}}} {e}^{- {a}_{l} m} ( {e}^{{a}_{l} ( {n}_{0} + {N}_{2} )} - {e}^{{a}_{l} {n}_{0}} )\end{matrix} & ( {n}_{0} + {N}_{2} < m )\end{matrix} & ( 19 )\end{matrix}$$

Therefore, when such an analytical solution is stored in the tube bulb information storing unit 521, it is possible to obtain the output tube voltage without performing a convolution operation.

In some cases, other waveforms can also be obtained algebraically through use of, for example, the Laplace transform. Further, the model to be used in the fourth acquisition example of acquiring the tube voltage waveform is not limited to Expression (15), and another model can be used. For example, a model in which the impulse response is approximated by an n-th order polynomial expression may be used (in this case, the parameter is a coefficient of a polynomial expression). However, when another model is used, the parameter of the impulse response may not always be obtained only through the fitting as shown in Expression (17), and in some cases, a Laplace transform, a z-transform, or a Fourier transform may be required. Further, the model is not limited to a model in actual time, and for example, a model in a frequency space after the Fourier transform, a z-space in the z-transform, or an s-space after the Laplace transform may be assumed.

Further, in the fourth acquisition example of acquiring the tube voltage waveform, the step function is input to obtain the impulse response of the radiation tube bulb from its response, but the impulse response may be obtained directly by inputting an impulse function instead and subjecting its response to the fitting.

Unlike the processing of the first acquisition example of acquiring the tube voltage waveform described above, the processing from the second acquisition example of acquiring the tube voltage waveform described above to the fourth acquisition example of acquiring the tube voltage waveform allows the transient response to be calculated from the information provided in advance at the time of calibration, and is therefore employed as an effective method when it is difficult to measure the tube voltage or another such information on the spectrum of the radiation 101 during the acquisition of a radiation image. Further, in the fourth acquisition example of acquiring the tube voltage waveform, the calibration is performed before the radiation imaging, but may be performed after the radiation imaging. In general, the calibration is performed at a time other than the time of radiation application for image formation.

### Second Embodiment

Next, a second embodiment of the present invention is described. In the following description of the second embodiment, descriptions of the same items as those described above in the first embodiment are omitted, and items different from those described above in the first embodiment are described.

A schematic configuration of a radiation imaging apparatus according to the second embodiment is the same as the schematic configuration of the radiation imaging apparatus 100 according to the first embodiment illustrated in FIG. 1A. In addition, the circuit configuration of the radiation detector 120 illustrated in FIG. 1A and the operation method therefor are the same as the circuit configuration of the radiation detector 120 and the operation method therefor according to the first embodiment, which are illustrated in FIG. 2, FIG. 3A, and FIG. 3B.

In the first embodiment described above, the spectrum of the radiation 101 is calculated by obtaining the tube voltage waveform. Meanwhile, in the second embodiment, the spectrum of the radiation 101 is measured without obtaining the tube voltage waveform, and the measured spectrum of the radiation 101 is associated with the imaging conditions, to thereby directly detect the spectrum of the radiation 101. In this case, the transient response of the radiation tube bulb being the radiation generator 110 is taken into consideration when the spectrum of the radiation 101 is measured.

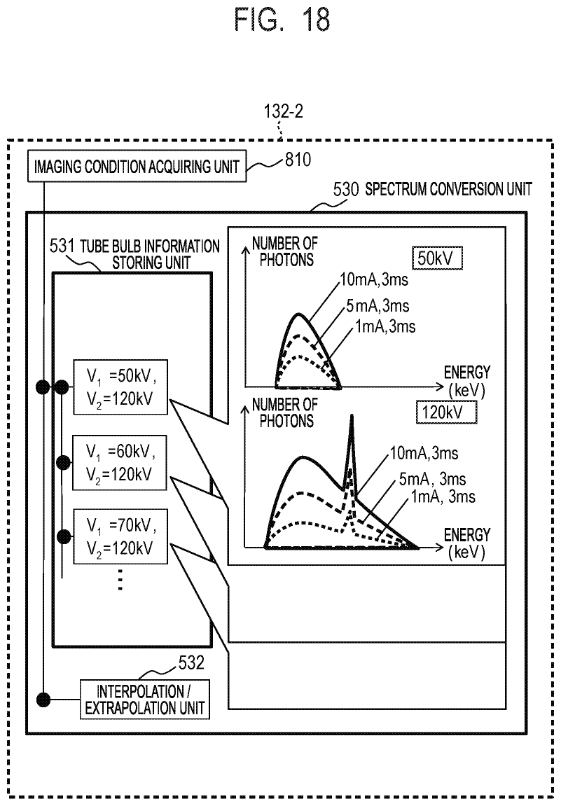

FIG. 18 is a diagram for illustrating an example of a functional configuration of the spectrum calculating unit 132 illustrated in FIG. 1A in the second embodiment of the present invention. In this case, the spectrum calculating unit 132 illustrated in FIG. 18 is referred to as “spectrum calculating unit 132-2”. In FIG. 18, the same components as those illustrated in FIG. 5, FIG. 8, FIG. 11, and FIG. 15 are denoted by the same reference symbols, and detailed descriptions thereof are omitted.

The spectrum calculating unit 132-2 illustrated in FIG. 18 includes functional components of the imaging condition acquiring unit 810 and the spectrum conversion unit 530. The spectrum conversion unit 530 includes a tube bulb information storing unit 531 corresponding to the tube bulb information storing unit 521 in the first embodiment and an interpolation/extrapolation unit 532 corresponding to the interpolation/extrapolation unit 523 in the first embodiment.

The imaging condition acquiring unit 810 acquires imaging conditions relating to radiation imaging of the radiation generator 110. The spectrum conversion unit 530 calculates the spectrum of the radiation 101 through conversion by processing the imaging conditions acquired by the imaging condition acquiring unit 810 based on the transient response characteristic of the radiation generator 110, which is stored in the tube bulb information storing unit 531.

The tube bulb information storing unit 531 stores information on the transient response characteristic of the radiation tube bulb being the radiation generator 110. Specifically, the tube bulb information storing unit 531 stores the spectrum of the radiation 101 (histogram having the horizontal axis representing the energy of one photon and the vertical axis representing the number of photons) associated with calibration conditions (set tube voltage, set tube current, set pulse width, radiation tube bulb temperature, and heat unit of the radiation tube bulb) as the transient response characteristic of the radiation generator 110. As the spectrum at this time, a spectrum corresponding to the low-energy first tube voltage V₁ and a spectrum corresponding to the high-energy second tube voltage V₂ are stored. When the value of the first tube voltage V₁ is different even at the same second tube voltage V₂, the shape of the spectrum of the second tube voltage V₂ changes. Therefore, when the first tube voltage V₁ is different, the spectrum of the second tube voltage V₂ at each first tube voltage V₁ may be held.

The interpolation/extrapolation unit 532 calculates the spectrum of the radiation 101 through conversion by performing the interpolation or extrapolation based on the imaging conditions acquired by the imaging condition acquiring unit 810 through use of the transient response characteristic of the radiation generator 110, which is stored in the tube bulb information storing unit 531.

In the first embodiment described above, the tube voltage deriving unit 520 includes the tube bulb information storing unit 521. However, in the second embodiment, it is not required to derive the tube voltage, and hence the tube voltage deriving unit 520 is not included. Therefore, the spectrum conversion unit 530 in the second embodiment includes the tube bulb information storing unit 531 corresponding to the tube bulb information storing unit 521 in the first embodiment.

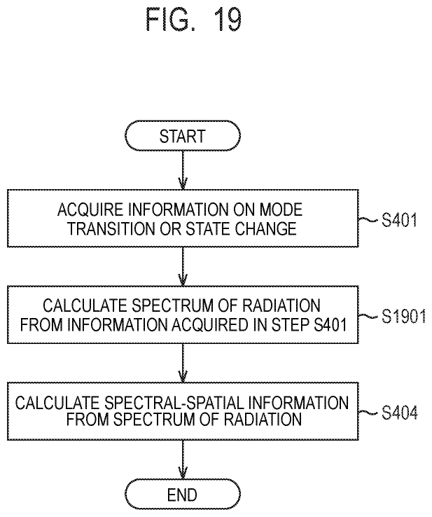

FIG. 19 is a flow chart for illustrating an example of a processing procedure of the spectrum calculating unit 132-2 illustrated in FIG. 18. In FIG. 19, the same processing steps as those illustrated in FIG. 4 are denoted by the same step numbers, and detailed descriptions thereof are omitted.

First, in Step S401 of FIG. 19, when detecting a mode transition or a state change, the spectrum calculating unit 132-2 acquires information on the detected mode transition or the detected state change.

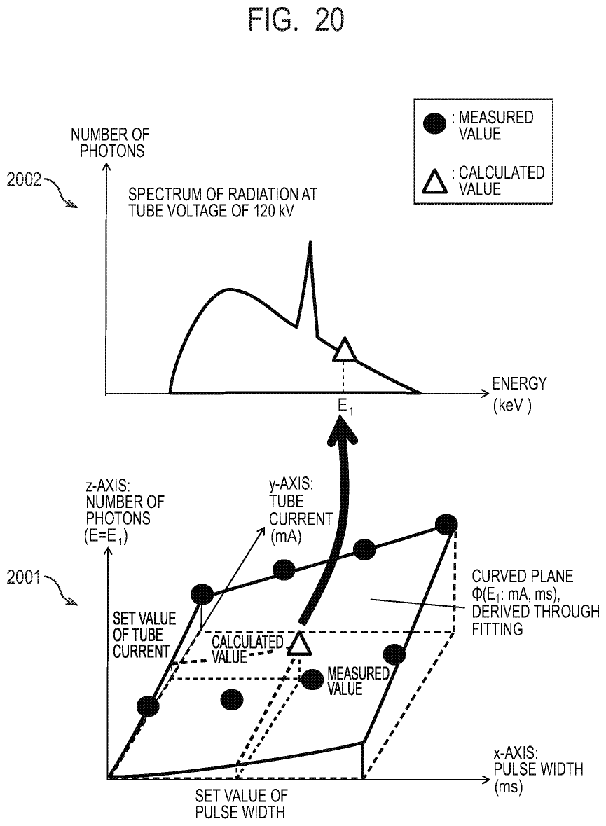

Subsequently, in Step S1901, the spectrum calculating unit 132-2 calculates the spectrum of the radiation 101 based on the information acquired in Step S401. In the second embodiment, the spectrum is not only the function of the energy of one photon but also the function of the tube current and the input pulse width. In this case, the spectrum calculating unit 132-2 fixes the tube voltage, acquires pieces of data at a plurality of input pulse widths or tube currents, and plots the acquired pieces of data in such a three-dimensional graph having the x-axis representing the pulse width, the y-axis representing the tube current, and the z-axis representing the number of photons as shown in a display area 2001 of FIG. 20 (measured values (black points) in the display area 2001 of FIG. 20). Then, the spectrum calculating unit 132-2 performs two-dimensional fitting on a function Φ(E: mA, ms) (where E represents the energy of one photon, mA represents the tube current, and ms represents the pulse width) being a curved plane with respect to those points, to thereby obtain the number of photons for a freely-selected tube current and a freely-selected pulse width. Then, the spectrum calculating unit 132-2 holds the function Φ(E: mA, ms) for each energy E, and the spectrum of the radiation 101 can be obtained from the acquired imaging conditions as shown in a display area 2002 of FIG. 20.

A portion having a long input pulse width and a large tube current exhibits no input pulse width dependency and no tube current dependency. The data acquisition is omitted in the portion having such an input pulse width and such a tube current.

In the second embodiment, when the first tube voltage V₁ is different even at the same second tube voltage V₂, the spectrum of the second tube voltage V₂ at each first tube voltage V₁ is held. When it is not required to take the accuracy into consideration, data may be shared for the same second tube voltages V₂. Meanwhile, even when the second tube voltage V₂ is different at the same first tube voltage V₁, the spectrum of the first tube voltage V₁ at each second tube voltage V₂ may be held.

Further, the measured spectrum is acquired through use of the spectrometer provided to the measuring device 160. Then, the acquired spectrum is stored in the tube bulb information storing unit 531 in association with the first tube voltage V₁, the second tube voltage V₂, the tube current, and the pulse width.

Unlike the first acquisition example of acquiring the tube voltage waveform in the first embodiment, the second embodiment allows the transient response to be calculated from the information provided in advance at the time of calibration, and is therefore employed as an effective method when it is difficult to measure the tube voltage or another such information on the spectrum of the radiation 101 during the acquisition of a radiation image. Further, the calibration is performed before the radiation imaging, but may be performed after the radiation imaging. In general, the calibration is performed at a time other than the time of radiation application for image formation.

According to the first and second embodiments described above, it is possible to calculate the spectrum of a radiation with high accuracy.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

What is claimed is:

1. A radiation imaging apparatus configured to image an imaging target object through use of radiation generated by a radiation generator arranged to generate the radiation, the radiation imaging apparatus comprising:
a spectrum calculating unit configured to calculate a spectrum of the radiation based on a transient response characteristic of the radiation generator.

2. The radiation imaging apparatus according to claim 1, further comprising:
a radiation detector arranged at a position opposed to the radiation generator and arranged to detect the radiation generated by the radiation generator; and
a spatial information calculating unit configured to calculate spatial information on the spectrum based on an image obtained through the detection by the radiation detector and the spectrum calculated by the spectrum calculating unit.

3. The radiation imaging apparatus according to claim 1, wherein the spectrum calculating unit includes:
a voltage deriving unit configured to derive a voltage waveform of the radiation generator based on the transient response characteristic of the radiation generator; and
a spectrum conversion unit configured to calculate the spectrum through conversion based on the voltage waveform.

4. The radiation imaging apparatus according to claim 3, further comprising a waveform acquiring unit configured to acquire the voltage waveform of the radiation generator,
wherein the voltage deriving unit is configured to derive the voltage waveform of the radiation generator based on the voltage waveform acquired by the waveform acquiring unit.

5. The radiation imaging apparatus according to claim 4, wherein the voltage deriving unit includes:
a storage unit configured to store the transient response characteristic of the radiation generator; and
a waveform processing unit configured to process the voltage waveform acquired by the waveform acquiring unit through use of the transient response characteristic of the radiation generator, to thereby derive the voltage waveform of the radiation generator.

6. The radiation imaging apparatus according to claim 5, wherein the waveform processing unit includes:
a smoothing unit configured to perform processing for smoothing the voltage waveform acquired by the waveform acquiring unit; and
a waveform averaging unit configured to average a plurality of voltage waveforms processed by the smoothing unit with respect to a plurality of voltage waveforms acquired in time series by the waveform acquiring unit.

7. The radiation imaging apparatus according to claim 3, further comprising an imaging condition acquiring unit configured to acquire imaging conditions relating to the imaging of the radiation generator,
wherein the voltage deriving unit is configured to derive the voltage waveform of the radiation generator based on the transient response characteristic of the radiation generator and the imaging conditions acquired by the imaging condition acquiring unit.

8. The radiation imaging apparatus according to claim 7, wherein the voltage deriving unit includes:
a storage unit configured to store the transient response characteristic of the radiation generator; and
an interpolation/extrapolation unit configured to derive the voltage waveform of the radiation generator by performing interpolation or extrapolation based on the imaging conditions acquired by the imaging condition acquiring unit through use of the transient response characteristic.

9. The radiation imaging apparatus according to claim 7,
wherein the transient response characteristic includes a characteristic of an impulse response, and
wherein the voltage deriving unit includes an impulse response calculating unit configured to perform calculation relating to the impulse response.

10. The radiation imaging apparatus according to claim 8,
wherein the storage unit is configured to store a response model relating to the transient response characteristic, and
wherein the voltage deriving unit is configured to derive the voltage waveform of the radiation generator through use of the response model.

11. The radiation imaging apparatus according to claim 10, wherein the voltage deriving unit further includes a fitting unit configured to perform processing for fitting relating to the response model.

12. The radiation imaging apparatus according to claim 1, further comprising an imaging condition acquiring unit configured to acquire imaging conditions relating to the imaging of the radiation generator,
wherein the spectrum calculating unit includes a spectrum conversion unit configured to calculate the spectrum through conversion by processing the imaging conditions acquired by the imaging condition acquiring unit based on the transient response characteristic of the radiation generator.

13. The radiation imaging apparatus according to claim 12, wherein the spectrum conversion unit includes:
a storage unit configured to store the transient response characteristic of the radiation generator; and
an interpolation/extrapolation unit configured to calculate the spectrum through conversion by performing interpolation or extrapolation based on the imaging conditions acquired by the imaging condition acquiring unit through use of the transient response characteristic of the radiation generator.

14. An operation method for a radiation imaging apparatus configured to image an imaging target object through use of a radiation generated by a radiation generator arranged to generate the radiation, the operation method comprising:
calculating a spectrum of the radiation based on a transient response characteristic of the radiation generator.

15. A non-transitory computer-readable medium having stored thereon a program for causing, when executed by a computer, the computer to execute an operation method for a radiation imaging apparatus configured to image an imaging target object through use of a radiation generated by a radiation generator arranged to generate the radiation, the operation method including:
calculating a spectrum of the radiation based on a transient response characteristic of the radiation generator.
